(19) United States
(12) Patent Application Publication
(10) Pub. No.: US 2026/0090584 A1
(43) Pub. Date: Apr. 2, 2026

(54) SYSTEMS AND METHODS FOR ESTIMATING AEROSOLIZATION EMISSION

(71) Applicant: SCIENTIFIC HORIZONS CONSULTING LLC, Irvine, CA (US)

(72) Inventors: Xiang GAO, Irvine, CA (US); Yatao LIU, Irvine, CA (US)

(73) Assignee: SCIENTIFIC HORIZONS CONSULTING LLC, Irvine, CA (US)

(21) Appl. No.: 19/113,241

(22) PCT Filed: Sep. 22, 2023
(86) PCT No.: PCT/US2023/074889

§ 371 Date: Mar. 19, 2025

Related U.S. Application Data
(60) Provisional application No. 63/409,324, filed on Sep. 23, 2022.

(51) Int. Cl. A24F 40/53; A24F 40/10; A24F 40/20; A24F 40/65; A24F 40/80; G16H 20/13

(52) U.S. Cl. CPC A24F 40/53; A24F 40/10; A24F 40/20; A24F 40/65; A24F 40/80; G16H 20/13

(57) ABSTRACT

A controller includes a processor and a memory storing instructions configured to cause the processor to receive emission test data of a test aerosolization system corresponding to a test aerosolizer power or a range of test aerosolizer powers of a test aerosolizer of the test aerosolization system and a test concentration or a range of test concentrations, receive operation data of a user aerosolization system corresponding to an operation duration based at least in part on an input signal from a user, the operation data comprising at least a concentration of a consumable included in the user aerosolization system and a aerosolizer power level of a aerosolizer of the user aerosolization system during the operation duration, estimate a emission per operation duration based at least in part on the operation data and the emission test data, and generate a signal indicative of the estimated emission per operation duration.

### CROSS-REFERENCE

This application claims benefit of U.S. Provisional Patent Application No. 63/409,324 filed on Sep. 23, 2022, which is incorporated herein by reference in its entirety.

### BACKGROUND

Assessing emission from an aerosolization system and operation behavior can aid in understanding product application, abuse liability, and provide quantitative inhalation dosage assessment of aerosol emissions and consumptions. Typically, consumable emission and consumption and inhalation topography is assessed through (1) self-reporting survey, (2) frame-by-frame video recording, and/or (3) added inhalation recording sensors. Self-reporting survey relies on users to input their own information, which may result in inaccuracies or biased answers. Frame-by-frame video recording is an inconvenient and unnatural setting for a user and may result in assessed consumable emission and consumption and inhalation topography that does not correspond to realistic and practical user operation patterns. Adding inhalation recording sensors to aerosolization systems may be cumbersome, invasive, and/or may otherwise alter a user's operation patterns, producing results that do not correspond to realistic and practical user operation patterns. Thus, there is a need for a solution to accurately, noninvasively, and conveniently assessing consumable emissions and consumption from an aerosolization system that reflect real-world operation of an aerosolization system by a user.

### SUMMARY

In an aspect of the present disclosure is a controller comprising: a processor and a memory storing instructions configured to cause said processor to receive emission test data of a test aerosolization system (TAS) corresponding to at least a test aerosolizer power or a range of test aerosolizer powers of a test aerosolizer of said test aerosolization system and a test concentration or a range of test concentrations, receive operation data of a user aerosolization system (UAS) corresponding to an operation duration based at least in part on an input signal from a user, said operation data comprising at least a concentration of a consumable included in said user aerosolization system and an aerosolization power level of an aerosolizer of said user aerosolization system during said operation duration, estimate an emission per operation duration based at least in part on said operation data and said emission test data, and generate a signal indicative of said estimated emission per operation duration. In some embodiments, said processor is configured to estimate said emission per operation duration based at least in part on a test emission of said emission test data, a test concentration, a test operation duration of said emission test data, and one or more aerosolization generation conditions. In some embodiments, said processor is configured to estimate said emission per operation duration based at least in part on said concentration and said operation duration. In some embodiments, said processor is configured to estimate said emission per operation duration using said following equation:

$$\frac{\begin{matrix}{emission}_{operation duration UAS , i} = \\ {emisssion}_{TAS}\end{matrix}}{{concentration}_{TAS} \times operation {duration}_{TAS}} \times {concentration}_{UAS , i} \times operation {duration}_{UAS , i}$$

wherein said emission_{operationduration UAS,i} is said emission per operation duration for an operation duration i, emission_{TAS} is said test emission, concentration_{TAS} is said test concentration, operation duration_{TAS} is said test operation duration, concentration_{UAS, i} is said concentration for said operation duration i, and operation duration_{UAS} is said operation duration i. In some embodiments, said processor is further configured to estimate a total emission of said aerosolization system based at least in part on a emission per operation duration and a total number of operation durations since an initial operation of said user aerosolization system by said user. In some embodiments, said processor is further configured to estimate an accumulated consumption over a period of time using said following equation:

$$accumulated {consumption}_{t 0 \to t 1} = {\sum }_{{operation}_{UAS , i} : t 0 \to t 1} consumable {emission}_{{operation}_{UAS , i}}$$

In some embodiments, said processor is further configured to estimate a consumable absorption in human based using said following equation:

$$consumable absorption in human {body}_{t 0 \to t 1} = Coefficient C \times accumulated {consumption}_{t 0 \to t 1}$$

wherein Coefficient C is a value in a range of approximately 0.001 to 0.80, inclusive.

In some embodiments, said instructions further cause said processor to display operation information on a user device, wherein said operation information comprises at least said emission per operation duration. In some embodiments, the controller further comprises a network interface communicably coupled to a communication network, wherein said communication network is communicably coupled to said user aerosolization system. In some embodiments, said communication network is communicably coupled to at least one database, wherein said at least one database stores said emission test data. In some embodiments, said operation duration corresponds to a duration of said input signal. In some embodiments, said input signal corresponds to activation of a switch included in said user aerosolization system, and said operation duration corresponds to an amount of time said switch is activated by said user. In some embodiments, said input signal corresponds to draw pressure from a pressure sensor, and the operation duration corresponds to an amount of time said draw pressure remains above a predetermined threshold. In some embodiments, said user aerosolization system is a nicotine aerosolization system.

In another aspect of the present disclosure is a method for determining an aerosolization emission, comprising receiving, by a controller, emission test data of a test aerosolization system (TAS) based at least in part on a test aerosolizer power or a range of a test aerosolizer powers of a test aerosolizer of said test aerosolization system and a test concentration or a range of test concentrations, receiving, by said controller, operation data of a user aerosolization system (UAS) corresponding to an operation duration based at least in part on an input signal from a user, said operation data comprising at least a concentration of a consumable included in said user aerosolization system and an aerosolizer power level of an aerosolizer of said user aerosolization system during said operation duration, estimating, by said controller, a emission per operation duration based at least in part on said operation data and emission test data, and generating, by said controller, a signal indicative of said estimated emission per operation duration. In some embodiments, estimating said emission per operation duration is based at least in part on a test emission of said emission test data, a test concentration, a test operation duration of said emission test data, and one or more aerosolization general conditions.

In some embodiments, estimating said emission per operation duration is based at least in part on said concentration and said operation duration. In some embodiments, said controller is configured to estimate said emission per operation duration using said following equation:

$$\frac{\begin{matrix}{emission}_{operation duration UAS , i} = \\ {emisssion}_{TAS}\end{matrix}}{{concentration}_{TAS} \times operation {duration}_{TAS}} \times {concentration}_{UAS , i} \times usage {duration}_{UAS , i}$$

wherein said emission_{operationduration UAS, i} is said emission per operation duration for an operation duration i, emission_{TAS} is said test emission, concentration_{TAS} is said test concentration, operation duration_{TAS} is said test operation duration for said operation duration i, concentration_{UAS} is said concentration, and operation duration_{UAS} is said operation duration i. In some embodiments, the method further comprises estimating a total emission of said aerosolization system based at least in part on a number of emission per operation duration and a total number of operation durations since an initial operation of said user aerosolization system by said user. In some embodiments, the method further comprises estimating an accumulated consumption over a period of time using said following equation:

$$accumulated {consumption}_{t 0 \to t 1} = {\sum }_{{operation}_{UAS , i} : t 0 \to t 1} consumable {emission}_{{operation}_{UAS , i}}$$

In some embodiments, the method further comprises estimating a consumable absorption in human based using said following equation:

$$consumable absorption in human {body}_{t 0 \to t 1} = Coefficient C \times accumulated {consumption}_{t 0 \to t 1}$$

wherein Coefficient C is a value in a range of approximately 0.001 to 0.80, inclusive. In some embodiments, said signal further indicates said total emission of said aerosolization system. In some embodiments, said operation duration corresponds to a duration of said input signal.

In an aspect of the present disclosure is an aerosolization device, comprising an aerosolizer configured to aerosolize a consumable, and a controller comprising (i) one or more processors and (ii) one or more memories storing computer-executable instructions that, when executed, cause said one or more processors to (a) receive operation data of said aerosolizer correspond to an operation duration based at least in part on an input signal from a user, said operation data comprising at least a concentration of said consumable included in said aerosolizer and an aerosolization power level of said aerosolizer during said operation duration, (b) estimate an emission per operation duration for said aerosolizer based at least in part on said operation data, and (c) cause said aerosolizer to suspend aerosolization of said consumable based at least in part on said emission per operation duration. In some embodiments, said emission per operation duration is estimated based at least in part on said concentration and said operation duration. In some embodiments, said computer-executable instructions, when executed, further cause said one or more processors to estimate a total emission of said aerosolizer based at least in part on an emission per operation duration and a total number of operation durations since an initial operation of said aerosolizer by said user. In some embodiments, said computer-executable instructions, when executed, further cause said one or more processors to estimate an accumulated consumption over a period of time using said following equation:

$$accumulated {consumption}_{t 0 \to t 1} = {\sum }_{{operation}_{UAS , i} : t 0 \to t 1} consumable {emission}_{{operation}_{UAS , i}}$$

In some embodiments, said computer-executable instructions, when executed, further cause said one or more processors to estimate a consumable absorption in human based using said following equation:

$$consumable absorption in human {body}_{t 0 \to t 1} = Coefficient C \times accumulated {consumption}_{t 0 \to t 1}$$

wherein Coefficient is a value in a range of approximately 0.001 to 0.80, inclusive. In some embodiments, said computer-executable instructions, when executed, further cause said one or more processors to display operation information on a user device, wherein said operation information comprises at least said emission per operation duration. In some embodiments, the aerosolization device further comprises a network interface communicably coupled to a communication network, wherein said communication network is communicably coupled to said aerosolizer. In some embodiments, said communication network is communicably coupled to at least one database, wherein said at least one database stores emission test data. In some embodiments, said operation duration corresponds to a duration of said input signal. In some embodiments, said input signal corresponds to activation of a switch included in said aerosolizer, and said operation duration corresponds to an amount of time said switch is activated by said user. In some embodiments, said input signal corresponds to draw pressure from a pressure sensor, and said operation duration corresponds to an amount of time said draw pressure remains above a predetermined threshold.

In some embodiments, said aerosolizer is a nicotine aerosolization system. In some embodiments, said consumable comprises one or more of 5-MeO-DMT (5-methoxy-N,N-dimethyltryptamine), Psilocybin, MDMA (3,4-Methylenedioxymethamphetamine), DMT (N,N-Dimethyltryptamine), LSD (Lysergic acid diethylamide), Ketamine or esketamine. In some embodiments, said consumable comprises one or more of 4-isobutyl-alpha-methylphenylacetic acid (IBUPROFEN®), acetylsalicylic acid (ASPIRIN®), (S)-(+)-2-(6-methoxy-2-naphthyl)propionic acid (NAPROXEN®), hydrocortisone, diphenhydramine (BENADRYL®), chlorpheniramine maleate (CLARITIN®), doxylamine succinate (UNISOM®), cetirizine dihydrochloride, melatonin, 1-tryptophan, 5-hydroxy-1-tryptophan, 4-acetamidophenol (TYLENOL®), 1-phenylephrine, guaiacol glycerol ether (MUCINEX®), salbutamol hemisulfate, humic acid, or other medicaments. In some embodiments, said consumable comprises one or more of phosphatidylcholine, cranberry powder, echinacea extract, feverfew extract, flaxseed extract, flaxseed extract, honeysuckle extract, white willow bark extract, lotus leaf extract, organic ginger extract, ashwagandha, bilberry, hops, horse chestnut, green coffee bean, luteolin, milk thistle, olive leaf, Radix isatidis, Rhodiola rosea, rose hip, tongkat ali, quercetin, American ginseng, Andrographis, astragalus, black pepper extract (95% piperine), boswelia serrata, cassia seed, chamomile, cinnamon bark, dandelion, dong quai root, elderberry, fennel seed, fenugreek, Ginkgo biloba, ginseng root, gotu kola, green tea extract (50% EGCG), green tea extract (50% polyphenols), hawthorn berry, lemon balm, lemon powder, licorice root, luo han guo, magnolia, marshmallow root, papaya fruit, passion flower, peppermint, pine bark, pomegranate, red clover, resveratrol, rhodiola extract (3% salidroside), schisandra, slippery elm bark, spirulina, tart cherry, theobromine, tribulus terrestris, turmeric extract (95% curcuminoids), valerian root, white mulberry, wild cherry, or wild jujube. In some embodiments, said consumable comprises one or more of Agomelatine, Duloxetine, Imipramine, or other depression medications. In some embodiments, said consumable comprises one or more of Apomorphine, Levodopa, or other Parkinson's medication. In some embodiments, said consumable comprises one or more of Loxapine, Chlorpromazine, or other schizophrenic or bipolar disorder medications. In some embodiments, said consumable comprises one or more of Dihydroergotamine, Sumatriptan, Prochlorperazine, Metoclopramide, Lidocaine, Duloxetine, or other migraine medications. In some embodiments, said consumable comprises one or more of caffeine, tea, or other stimulants. In some embodiments, said consumable comprises one or more of Ciprofloxacin, Norfloxacin, Balofloxacin, Rufloxacin, Fleroxacin, Gatifloxacin, Levofloxacin, Moxifloxacin, Ofloxacin, Sparfloxacin, Pefloxacin, Nadifloxacin, Clofazimine, Homosulfamine, Metronidazole, Ampicillin, Azithromycin, Tetracycline, Vancomycin, Amikacin, Cefadroxil, Aztreonam, Tobramycin, or other antibiotics.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic block diagram of an emission system, according to an embodiment.

FIG. 2 is a schematic block diagram of a controller that may be included in the emission system of FIG. 1, according to an embodiment.

FIG. 3A is a schematic flow chart of a method for estimating a consumable emission per operation duration, according to an embodiment.

FIG. 3B is a schematic flow chart of another method for estimating consumable emission as well as estimate total consumable emission over a period of time and amount of consumable absorbed in a user's body over the period of time, according to an embodiment.

FIGS. 4A-4C depict box plots of puff topography parameters measured by an exemplary puff recording nicotine delivery system (PR-ENDS).

FIGS. 5A-5C depict box plots of puff topography parameters measured by a Clinical Research Support System (CReSS) and an exemplary PR-ENDS device.

FIGS. 6A-6C depict correlations of measured values between CReSS and PR-ENDS.

FIG. 7 depicts a comparison of box plots of nicotine consumption within on-hour ad-libitum session between: (1) value estimated based at least in part on puff topography measured by a PR-ENDS device; and (2) value estimated based at least in part on e-liquid weight loss among smokers and vapers.

FIG. 8 depicts nicotine plasma concentration-time profile over 60 minutes by product group among smokers and vapers.

FIG. 9 depicts a linear regression of: (1) the plasma concentration-time curve and the PR-ENDS derived nicotine consumption; and (2) the plasma concentration-time curve and e-liquid weight loss derived nicotine consumption among smokers and vapers.

FIGS. 10A-10B depict PR-ENDS distributions by device power and by e-liquid nicotine concentration.

FIG. 11A depicts the distribution of PR-ENDS puff duration.

FIG. 11B depicts a comparison of puff duration by device power, e-liquid nicotine concentration, cigarette smoking history, and PR-ENDS use history.

FIGS. 12A-12C depict linear regression plotting between nicotine emission per puff and e-liquid nicotine concentrations among high, medium, and low device powers.

FIG. 13A depicts the distribution of PR-ENDS nicotine emission.

FIG. 13B depicts a comparison of nicotine emission (mg/puff) by device power, e-liquid nicotine concentration, cigarette smoking history, and PR-ENDS use history.

FIG. 14A depicts a daily histogram representation of puff number for a group of participants.

FIG. 14B depicts participant distribution of their coefficient of variance in daily puff duration, puffs per day, and daily nicotine consumption over the active period.

FIGS. 15A-15F depict observations of puffs per day during the actual use of PR-ENDS over one, two, four, five, six, and seven weeks.

FIG. 16A-16C depict observations of puffs per day, puff duration per day, and nicotine consumption per day of the user group during the actual use of PR-ENDS over three weeks.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Embodiments disclosed herein relate to systems and methods for estimating emission of a consumable (e.g., nicotine, cannabis, etc.). In particular, embodiments disclosed herein relate to a controller including a processor and a memory. The memory stores instructions configured to cause the processor to receive emission test data (e.g., consumable emission test data) of a test aerosolization system corresponding to a test aerosolizer power or a range of test aerosolizer powers of a test aerosolizer of the test aerosolization system and a test concentration (e.g., test consumable concentration) or a range of test concentrations (e.g., test consumable concentrations). The memory further stores instructions configured to cause the processor to receive operation data of a user aerosolization system corresponding to an operation duration based at least in part on an input signal from a user, the operation data comprising at least a concentration (e.g., consumable concentration) of a consumable included in the user aerosolization system and an aerosolizer power level of an aerosolizer of the user aerosolization system during the operation duration. The memory further stores instructions causing the processor to estimate an emission per operation duration (e.g., consumable emission per operation duration) based at least in part on the operation data and the consumable emission test data and to generate a signal indicative of the estimated consumable emission per operation duration. In some embodiments, the test aerosolization system and the user aerosolization system may be the same system. In other embodiments, the test aerosolization system and the user aerosolization system may be similar systems but may not be the exact same system. For example, the test aerosolization system and the user aerosolization system may be the same model, include the same components, include components with the same capabilities, and/or the like. In some embodiments, the user aerosolization system may be a nicotine aerosolization system.

In some embodiments, the processor may be configured to estimate the consumable emission per operation duration based at least in part on a test emission (e.g., test consumable emission) of the consumable emission test data, a test concentration (e.g., test consumable concentration), and a test operation duration of the emission test data. In some embodiments, the processor may be configured to estimate the consumable emission per operation duration based at least in part on the consumable concentration and the operation duration. For example, the processor may be configured to estimate the consumable emission per operation duration using equation (1):

$$\frac{\begin{matrix}consumable {emission}_{operation duration UAS , i} = \\ consumable {emission}_{TAS}\end{matrix}}{consumable {concentration}_{TAS} \times operation {duration}_{TAS}} \times consumable {concentration}_{UAS , i} \times operation {duration}_{UAS , i} \qquad ( 1 )$$

wherein the consumable emission_{operation duration UAS, i} is the consumable emission per operation duration for an operation duration i, consumable emission_{TAS} is the test consumable emission, consumable concentration_{TAS} is the test consumable concentration, operation duration_{TAS} is the test operation duration, consumable concentration_{UAS, i} is the consumable concentration for the operation duration i, and operation duration_{UAS} is the operation duration i.

In some embodiments, the processor may be configured to estimate an accumulated consumption over a period of time using equation (2):

$$accumulated consumable {consumption}_{t 0 \to t 1} = {\sum }_{{usage}_{UAS , i} : t 0 \to t 1} consumable {emission}_{{usage}_{UAS , i}} \qquad ( 2 )$$

In some embodiments, the processor may be configured to estimate a consumable absorption in a human using the equation (3):

$$consumable absorption in human {body}_{t 0 \to t 1} = Coefficient C \times accumulated {consumption}_{t 0 \to t 1} \qquad ( 3 )$$

where Coefficient C is a function of at least one of device aerosolization power, aerosol chemical and physical properties (e.g., aerosol particle size, aerosol particle surface charge, aerosol particle hydrophobicity, etc.). In some embodiments, Coefficient C has a value in a range of approximately 0.001 to 0.80, inclusive.

In some embodiments, the processor may be further configured to estimate a total emission (e.g., total consumable emission) of the aerosolization system based at least in part on an emission per operation duration (e.g., consumable emission per operation duration) and a total number of operation durations since an initial operation of the user aerosolization system by the user. In some embodiments, the processor may be further configured to display operation information on a user device in which the operation information includes at least the consumable emission per operation duration. In some embodiments, the controller may include a network interface communicably coupled to a communication network. The communication network may be communicably coupled to the user aerosolization system. In some embodiments, the communication network may be communicably coupled to at least one database, wherein the at least one database stores the consumable emission test data.

In some embodiments, the operation duration may correspond to a duration of the input signal. In some variations, the input signal may correspond to activation of a switch included in the user aerosolization system and the operation duration may correspond to an amount of time the switch is activated by the user. In other variations, the input signal may correspond to draw pressure from a pressure sensor and the operation duration may correspond to an amount of time the draw pressure remains above a predetermined threshold.

Advantageously, embodiments of the systems and the methods disclosed herein for estimating a consumable emission of an aerosolization system may provide one or more benefits. For example the systems and the method disclosed herein may allow for non-invasive estimation of consumable emission without affecting a user's habits. In another example, the systems and the methods disclosed herein may identify the complex interaction between aerosolization system settings and consumable concentrations during actual use and effects on behavior. In another example, the systems and the methods disclosed herein may allow for an identification of user behavior when using an aerosolization system over time. In another example, the systems and the methods disclosed herein may allow estimation of consumable emission in various aerosolization systems and devices, or for different consumables. In another example, the systems and the methods disclosed herein may enable estimation of accumulated consumption and consumable absorption in human body, thus allowing a user to use this information to modify or adjust user behavior to adjust consumable consumption. In another example, the systems and the methods disclosed herein may use an efficient process that reduces processor operating time and processor load, thereby reducing computing load and power draw and increasing power source life. In another example, the systems and the methods disclosed herein by using an efficient process that reduces processor operating time and processor load, may reduce network traffic in cases where data/information collected, generated, obtained, etc. by the systems and the methods disclosed herein is transmitted (e.g., over the Internet, over the cloud, over a router, over one or more communication protocols, etc.).

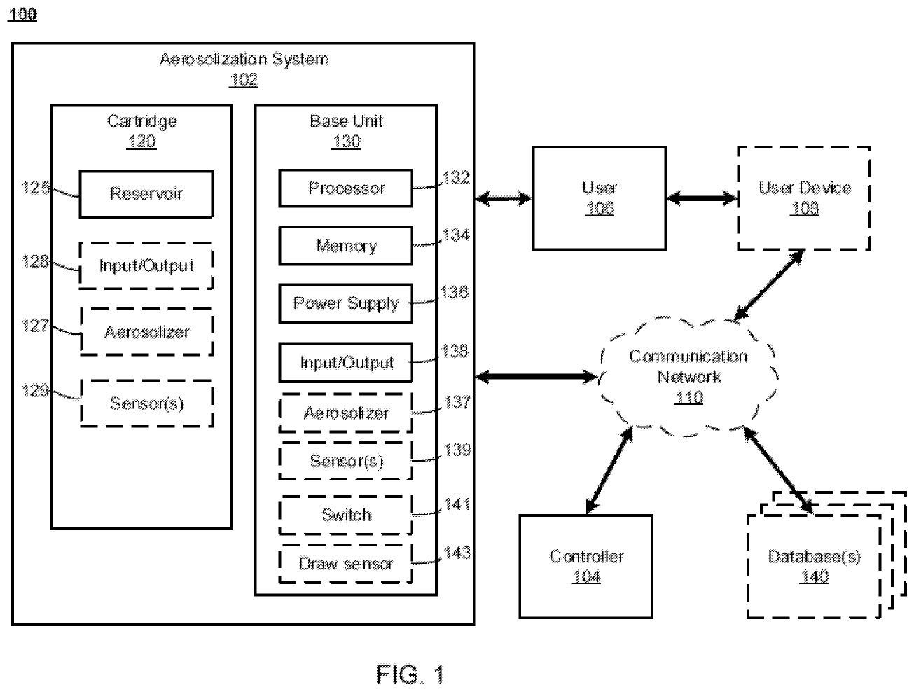

FIG. 1 is a schematic block diagram of an emission system 100, according to an embodiment. The emission system 100 includes an aerosolization system 102, a controller 104, and a user 106. In some embodiments, the emissions system 100 may include a user device 108, a communication network 110, and/or at least one database 140. The emission system 100 may be configured to determine consumable emission from the aerosolization system 102.

### Aerosolization System

The aerosolization system 102 may comprise a device configured to aerosolize solid and/or liquid consumables (e.g., vaporizer, vape, e-cigarette, heated tobacco product, e-cigar, e-pipe, mesh vibrating nebulizer, jet nebulizer, dry powder inhaler, metered dose inhaler, pressure metered dose inhaler, other nebulizer, and inhalers, etc.). In some embodiments, the aerosolization system may include a cartridge 120 coupled to a base unit 130. In some embodiments, the cartridge 120 and the base unit 130 may be integrally formed, while in other embodiments, the cartridge 120 and the base unit 130 may be formed separately and coupled to one another. In some embodiments, the cartridge 120 may be selectively removable from the base unit 130. The cartridge 120 may be manufactured, shipped, and/or sold separately from the base unit 130. A user, such as user 106, may assemble the aerosolization system 102 by mechanically connecting the cartridge 120 to the base unit 130 (e.g., via threads, a snap-fit, a friction-fit, magnets, or any other suitable coupling mechanism).

### Cartridge

The cartridge 120 may comprise a reservoir 125. The reservoir 125 may contain a liquid or solid consumable for aerosolization. The reservoir 125 may include additional components, such as a wick, to aid in generating an aerosol. The cartridge 120 may include an input/output interface 128. The input/output interface 128 may be configured to send and receive information regarding the cartridge 120, such as, for example, the contents of the reservoir 125. More specifically, in some variations, the input/output interface 128 may be configured to send the type of consumable, state (e.g., liquid, or solid) of the consumable, consumable concentration, and the like. The input/output interface 128 may be a scannable code, an NFC tag, a circuit, a Bluetooth module, or other device configured to send and/or receive information. The input/output interface 128 may also include an output device such as a screen, indicator light, audio transmitter, etc. that is configured to provide information regarding the cartridge 120. For example, the input/output interface 128 may include an indicator light that indicates when the amount of consumable in the reservoir 125 is below a predetermined threshold. In some embodiment, the cartridge 120 may include identifying information that may be visual (e.g., label, color, etc.) and/or may communicate with the base unit 130 to provide the base unit 130 with identifying information via an identifying characteristic (e.g., color, scannable code, magnetic strip, etc.).

In some embodiments, the cartridge 120 may further comprise an aerosolizer 127. Additionally or alternatively, the base unit 130 may comprise an aerosolizer 137. In some variations in which the base unit 130 comprises an aerosolizer 137, it may be unnecessary for the cartridge 120 to include an aerosolizer 127. In some embodiments, the aerosolizer 127 or 137 may include a heater (e.g., a resistive heater, a ceramic heater, a wound coil heater, an induction heater, any other suitable heater, or a combination thereof). In some embodiments, the aerosolizer 127 or 137 may include a nebulizer (e.g., a vibrating mesh nebulizer, an ultrasonic nebulizer, a jet nebulizer, any other suitable nebulizer, or a combination thereof). The aerosolizer 127 may be configured to receive power from a power source (e.g., power supply 136) to cause the aerosolizer 127 to aerosolize the consumable. The aerosolizer 127 (e.g., a heating element of the aerosolizer) may be fluidly coupled to the reservoir 125, such that energy (e.g., heat, change in pressure, change in gas flow, etc.) may be transferred to the reservoir 137 to aerosolize the consumable contained therein and allow for the consumable to be inhaled by a user 106. The cartridge 120 may further include at least one sensor 129 configured to detect and measure characteristics (e.g., consumable amount in reservoir 125, consumable type, consumable concentration, weight of consumable, etc.) of the cartridge 120 and its components. The sensor(s) 129 may be disposed within the reservoir 125 or may be in communication (e.g., fluid, mechanical, etc.) with the reservoir 125. The input/output 128 may be configured to send information from the sensor(s) 129 to a location outside of the cartridge 120.

Any suitable consumable may be stored in the reservoir 125. For example, the consumable may include a composition including an inhalable material. In some embodiments, the consumable may include a nicotine-based consumable. In some embodiments, the consumable may include a cannabis or marijuana-based consumable. In some embodiments, the consumable may include one or more of a herb, a supplement (e.g., carboxylic acid, phenols, flavonoids, or a medicament such as, for example, one or more of 4-isobutyl-alpha-methylphenylacetic acid (IBUPROFEN®), acetylsalicylic acid (ASPIRIN®), (S)-(+)-2-(6-methoxy-2-naphthyl)propionic acid (NAPROXEN®), hydrocortisone, diphenhydramine (BENADRYL®), chlorpheniramine maleate (CLARITIN®), doxylamine succinate (UNISOM®), cetirizine dihydrochloride, melatonin, 1-tryptophan, 5-hydroxy-1-tryptophan, 4-acetamidophenol (TYLENOL®), 1-phenylephrine, guaiacol glycerol ether (MUCINEX®), salbutamol hemisulfate, humic acid, and any other suitable medicament or a combination thereof. In some embodiments, the consumable may include a composition including an herb extract. In some embodiments, the herb extract may include one or more of phosphatidylcholine, cranberry powder, echinacea extract, feverfew extract, flaxseed extract, flaxseed extract, honeysuckle extract, white willow bark extract, lotus leaf extract, organic ginger extract, ashwagandha, bilberry, hops, horse chestnut, green coffee bean, luteolin, milk thistle, olive leaf, Radix isatidis, Rhodiola rosea, rose hip, tongkat ali, quercetin, American ginseng, Andrographis, astragalus, black pepper extract (95% piperine), boswelia serrata, cassia seed, chamomile, cinnamon bark, dandelion, dong quai root, elderberry, fennel seed, fenugreek, Ginkgo biloba, ginseng root, gotu kola, green tea extract (50% EGCG), green tea extract (50% polyphenols), hawthorn berry, lemon balm, lemon powder, licorice root, luo han guo (e.g., monk fruit extract), magnolia, marshmallow root, papaya fruit, passion flower, peppermint, pine bark, pomegranate, red clover, resveratrol, rhodiola extract (3% salidroside), schisandra, slippery elm bark, spirulina, tart cherry, theobromine, tribulus terrestris, turmeric extract (95% curcuminoids), valerian root, white mulberry, wild cherry, wild jujube, or other herbal extract. In some embodiments, the consumable may include one or more psychedelics. For example, the consumable may include one or more of 5-MeO-DMT (5-methoxy-N,N-dimethyltryptamine), Psilocybin, MDMA (3,4-Methylenedioxymethamphetamine), DMT (N,N-Dimethyltryptamine), LSD (Lysergic acid diethylamide), Ketamine or esketamine, or other psychedelics. In some embodiments, the consumable may include one or more depression medications. For example, the consumable may include one or more of Agomelatine, Duloxetine, Imipramine, or other depression medications. In some embodiments, the consumable may include one or more medications for Parkinson's disease. For example, the consumable may include one or more of Apomorphine, Levodopa, or other Parkinson's medications. In some embodiments, the consumable may include one or more medications for schizophrenic or bipolar disorder. For example, the consumable may include one or more of Loxapine, Chlorpromazine, or other schizophrenic or bipolar disorder medications. In some embodiments, the consumable may include one or more migraine medications. For example, the consumable may include one or more of Dihydroergotamine, Sumatriptan, Prochlorperazine, Metoclopramide, Lidocaine, Duloxetine, or other migraine medications. In some embodiments, the consumable may include one or more stimulants. For example, the consumable may include one or more of caffeine, tea, or other stimulants. In some embodiments, the consumable may include one or more antibiotics. For example, the consumable may include one or more of Ciprofloxacin, Norfloxacin, Balofloxacin, Rufloxacin, Fleroxacin, Gatifloxacin, Levofloxacin, Moxifloxacin, Ofloxacin, Sparfloxacin, Pefloxacin, Nadifloxacin, Clofazimine, Homosulfamine, Metronidazole, Ampicillin, Azithromycin, Tetracycline, Vancomycin, Amikacin, Cefadroxil, Aztreonam, Tobramycin, or other antibiotics.

### Base Unit

The base unit 130 may be configured to facilitate the general operation of the aerosolization system 102, which may include receiving inputs regarding aerosolization system 102 settings (e.g., power level, cartridge type, etc.) and operation requests, operating components of the aerosolization system 102, and processing and communicating information (e.g., operation data, etc.). As shown in FIG. 1, the base unit 130 may include a processor 132, a memory 134, a power supply 136, an input/output interface 138, the aerosolizer 137, and at least one sensor 139. In some variations, the base unit 130 need not include the aerosolizer 137 and one or more sensors 139.

The processor 132 may be configured to complete operations based at least in part on instructions stored on the memory 134. The processor 132 may be implemented as a general-purpose processor, an ASIC, one or more FPGAs, a DSP, a group of processing components, or other suitable electronic processing components. The processor 132 may be configured to send and receive signals to and from other components of the base unit 130 (e.g., memory 134, power supply 136, input/output 138, aerosolizer 137, sensor(s) 139, etc.) and the cartridge 120.

While illustrated as included in the base unit 130, in some embodiments, the processor 132 may be included directly (e.g., outside of the base unit 130) in the aerosolization system 102 or the device configured to aerosolize solid and/or liquid consumables. In some embodiments, in addition to or in alternative to generating signals, the processor 132 may perform (e.g., directly or indirectly) an action. For example, the processor 132 may suspend vaporization of the consumable, compute and report emissions, etc.

In some embodiments, the processor 132 (e.g., central processing units (CPUs), general purpose graphics processing units (GPGPUs), or quantum processing units (QPUs)) may carry out functions. For example, the processor 132 may optionally include a cache memory unit for temporary local storage of instructions, data, or computer addresses. In some embodiments, the processor 132 is configured to assist in execution of computer readable instructions. For example, one or more components of the aerosolization system 102 may provide functionality for the components depicted in FIG. 1 as a result of the processor 132 executing non-transitory, processor-executable instructions embodied in one or more tangible computer-readable storage media, such as the memory 134, etc. In some embodiments, the computer-readable media may store software that implements particular operations, and the processor 132 may execute the software.

The processor 132 may perform functions of various illustrative logical blocks, modules, and circuits described in connection with the examples disclosed herein and may be implemented or performed as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions of FIG. 1. A general purpose processor may be a microprocessor, but in the alternative, the processor may be any processor, controller, microcontroller, or state machine. In some embodiments, the processor 132 may also be implemented as a combination of computing devices, e.g., a combination of a DSP and a microprocessor, a plurality of microprocessors, one or more microprocessors in conjunction with a DSP core, or any other such configuration. The operations of the systems, the methods, the computer-readable media, and the techniques the methods, the techniques, the algorithms, etc. described in connection with the examples disclosed herein may be embodied directly in hardware, in a software module executed by the processor 132, or in a combination of the two.

The memory 134 may include a non-transitory, processor readable medium (e.g., RAM, NVRAM, ROM, Flash Memory, hard disk storage, etc.) that stores data and/or computer code for facilitating the various processes disclosed herein. Moreover, the memory 134 may be or may include tangible, non-transient volatile memory, or non-volatile memory. The memory 134 may store information and instructions regarding the operation of the aerosolization system 102. For example, the memory 134 may store instructions configured to cause the aerosolization system 102 to generate an aerosol from the consumable disposed in the reservoir 125 based at least in part on an input from the user 106. The memory 134 may further store information from various components of the aerosolization system 102 regarding the operation of the aerosolization 102, such as number of uses, operation duration, consumable concentration, consumable type, and on the like.

The power supply 136 may be configured to provide power to the aerosolization system 102 generally and to the components of the cartridge 120 and the base unit 130. The power supply 136 may be constantly operating or may be selectively operational based at least in part on switching (e.g., selective switching) of the aerosolization system 102 between a powered (e.g., “ON”) and unpowered (e.g., “OFF”) setting. The power supply 136 may supply power to the aerosolizer (either aerosolizer 127 or aerosolizer 137) when directed by the processor to allow for the aerosolizer to aerosolize the consumable. The power supply 136 may include multiple power settings (e.g., low, medium, and high) that allow for a varied amount of energy to be released by the aerosolizer, thus varying the amount of consumable aerosolized. The aerosolizer 127 or 137 may include any suitable aerosolizer, for example, a resistive aerosolizer (e.g., a metallic or ceramic aerosolizer), an inductive aerosolizer, a radiative aerosolizer, any other suitable aerosolizer, or a combination thereof.

The input/output interface 138 may be configured to send and receive signals. For example, an input device of the input/output interface 138 may be configured to receive mechanical inputs (e.g., vibration, switch toggle, etc.) and/or digital inputs (e.g., signals, etc.). In some embodiments, the input device may further include an electromagnetic connector(s) for charging and/or data communication. In some embodiments, the input device may include a switch (e.g., the switch 141) that, when pressed, sends a signal to the processor to generate aerosol for a period of time equivalent to the duration the switch is activated. An output device of the input/output interface 138 may be configured to display information regarding the aerosolization system. For example, the output device may include a battery indicator that indicates the battery level of the power supply 138. In some embodiments, the input/output interface 138 may include wired and/or wireless communication capabilities (e.g., Bluetooth, Wi-Fi, etc.) that allow the base unit 120 to communicate with other components within the emission system 100 and other components of the aerosolization system.

The aerosolizer 137 may be functionally and/or structurally the same as the aerosolizer 127 of the cartridge 120. In some embodiments, the base unit 130 may include an aerosolizer 137 that is configured to be in communication (e.g., fluidic communication and/or thermal communication) with the reservoir 125, or the consumable stored in the reservoir 125 (e.g., via a wick). The aerosolizer 137 may be configured to selectively aerosolize the consumable (e.g., via heating or nebulizing), when the cartridge 120 is coupled to the base unit 130. In such embodiments, the aerosolizer 127 of the cartridge 120 may be excluded. The at least one sensor 139 may measure information regarding the base unit 130 and/or the cartridge 120. For example the sensor(s) 139 may measure the amount of power in the power supply 136, the amount of consumable aerosolized, etc.

### Activation Mechanisms

In some variations, the aerosolization system, and more particularly, the base unit 120, may comprise an activation mechanism, activation of which may send a signal to the aerosolization system to aerosolize the consumable. In some embodiments, the duration the activation mechanism is activated corresponds to the duration or amount of consumable aerosolized.

In some embodiments, the activation mechanism may be or may include a user activated switch. For example, the base unit 120 may include a user activated switch 141 (e.g., an activation button) that the user may engage (e.g., depress) to cause aerosolization of the consumable. For example, the aerosolizer 137 (or 127) may be configured to be activated in response to a user activating the switch 141 (e.g., depressing the activation button). In such embodiments, the input signal provided by the user may correspond to activation of the switch 141 included in the user aerosolization system 102, and the operation duration may correspond to an amount of time the switch 141 is activated by the user.

In some embodiments, the activation mechanism may be or may include a sensor, such as a draw sensor. For example, the base unit 130 may include a draw sensor 143 that is coupled to the input/output interface 138, for example, a pressure sensor configured to measure a draw pressure (e.g., a suction pressure) applied by a user on the aerosolization system (e.g., the cartridge 120). In some variations, an operation duration may correspond to the amount of time the draw pressure remains above the predetermined threshold. For example, the aerosolizer 137 may be configured to be activated when the draw sensor 143 determines that a draw pressure is exerted on the draw sensor 143 (e.g., due to a suction or draw being applied by a user on a mouthpiece of the cartridge 120), which is above a predetermined threshold. In such embodiments, the input signal for activating the heart 137 may correspond to the draw pressure from the draw sensor 143, and the operation duration corresponds to an amount of time the draw pressure remains above the predetermined threshold.

### Controller

As mentioned above, the emission system 100 may comprise a controller 104 communicably coupled to the base unit 130 of the aerosolization system and configured to receive and process information from the base unit 130 of the aerosolization system 102. In some embodiments, the controller 104 may be on a dedicated device separate from the aerosolization system 102. In some embodiments, the controller 104 may be included in the aerosolization system 102. In some embodiments, the controller 104 may be included in the user device 108. The controller 104 may be configured to communicate through wired (e.g., LAN, etc.) or wireless (e.g., Bluetooth, Wi-Fi) communication with the base unit 130 of the aerosolization system 102, the user device 108, and/or the database(s) 140.

The controller 104 may be configured to receive consumable emission test data for a test aerosolization system (e.g., functionally and/or structurally similar to the aerosolization system 102). The controller 104 may receive the consumable emission test data from the database(s) 140, which may have prepopulated or prestored consumable emission test data for the test aerosolization system that includes a test aerosolizer power or a range of test aerosolizer powers of the test aerosolization system, and a test consumable concentration or a range of test consumable concentrations. The consumable emission test data may include at least a test consumable emission of the consumable emission test data, a test consumable concentration, a test operation duration of the consumable emission test data, and/or one or more aerosolization conditions (e.g., inhalation duration, inhalation frequency, inhalation volume, number of inhalations, etc.) of a test aerosolization system that is used to determine the consumable emission test data. In some embodiments, the controller 104 may receive raw data corresponding to test aerosolization system testing from the database(s) 140 (e.g., a clinical research report system) and may process the raw data. In some embodiments, the controller 104 may have a test setting in which the controller 104 may determine and store values corresponding to those found in a laboratory setting. For example, the controller 104 may receive data directly from a test aerosolization system and may determine a consumable emission, a consumable concentration, and an operation duration of a test aerosolization system.

The controller 104 may be configured to receive operation data of a user aerosolization system (e.g., structurally and/or functionally similar to the aerosolization system 102 or may be different therefrom). The operation data may include data corresponding to an operation duration based at least in part on an input signal from a user. For example, the operation data may correspond to the amount of time a switch was pressed to aerosolize the consumable, or a draw pressure was applied on the cartridge 102 as detected by the draw sensor. The operation data may further include information regarding the particular operation, such as consumable concentration, consumable type, etc. In some embodiments, the controller 104 may receive or determine consumable characteristics, such as, for example one or more of the consumable type and consumable concentration. of the consumable.

Based at least in part on at least the operation data and the consumable emission test data, the controller 104 may be configured to estimate consumable emission per operation duration for the operation duration corresponding to the operation data. In some embodiments, the controller 104 may be configured to estimate the consumable emission per operation duration based at least in part on a test consumable emission of the consumable emission test data, a test consumable concentration, and a test operation duration of the emission test data. In some embodiments, the controller may be configured to estimate the consumable emission per operation duration based at least in part on the consumable concentration and the operation duration. For example, in some variations, the controller 104 may be configured to estimate the consumable emission per operation duration using equation (1):

$$\frac{\begin{matrix}consumable {emission}_{operation duration UAS , i} = \\ consumable {emisssion}_{TAS}\end{matrix}}{consumable {concentration}_{TAS} \times operation {duration}_{TAS}} \times consumable {concentration}_{UAS , i} \times operation {duration}_{UAS , i} \qquad ( 1 )$$

where the consumable emission_{operation duration UAS, i} is the consumable emission per operation duration for an operation duration i, consumable emission_{TAS} is the test consumable emission, consumable concentration_{TAS} is the test consumable concentration, operation duration_{TAS} is the test operation duration, consumable concentration_{UAS, i} is the consumable concentration for the operation duration i, and operation duration_{UAS} is the operation duration i. In some embodiments, the consumable emission_{TAS} may be based at least in part on or include information on the various aerosolizer powers used to aerosolize the consumable using the test aerosolization system. In some embodiments, the consumable emission_{operation duration UAS, i} may also be based at least in part on the aerosolization power at which the aerosolization system 102 is operated. To account for the different power settings the consumable emission_{TAS} is obtained, or the user aerosolization system 102 is operated at, the consumable emission_{TAS} may be represented as a matrix in equation 1 to illustrate tested consumable emission at different power settings. In some embodiments, within each power setting or value of the aerosolizer, the consumable emission corresponding to the various aerosolizing powers may be represented by a linear regression model, that may be used in equation (1) to consumable emission_{operationduration UAS, i}.

In some embodiments, the controller 104 may also be configured to estimate an accumulated consumption over a period of time using equation (2):

$$accumulated consumable {consumption}_{t 0 \to t 1} = {\sum }_{{usage}_{UAS , i} t 0 \to t 1} consumable {emission}_{{usage}_{UAS , i}} \qquad ( 2 )$$

The accumulated consumable emission represents the amount of consumable that may be inhaled by the user if all the aerosol or vapor generated by the aerosolization system 102 between a first time period t0 and a second time period t1 is inhaled. is The accumulated consumable emission is based at least in part on consumable emission per operation duration, operation number, operation frequency, etc. The difference between t1 and t0 represents the total time the aerosolization system 102 is activated, or may represent any other time period (e.g., consumable consumption per hour, per day, per week, etc.). In use however, the total amount of aerosol generated by the aerosolization system 102 may not be the actual amount consumed by the user. For example, the user may exhale some amount of the consumable while inhaling, or some amount of consumable aerosol may be lost during inhalation because of, for example, a non-hermetic seal between the user's mouth and a mouthpiece of the cartridge 120. Moreover, the actual amount of consumable absorbed in the body of the user may be less than the amount of aerosol inhaled by the user. In some embodiments, the controller 104 may be configured to estimate a consumable absorption in the human body (e.g., the user's body) for the time period t0 to t1 using equation (3):

$$consumable absorption in human {body}_{t 0 \to t 1} = Coefficient C ⨯ accumulated consumable {consumption}_{t 0 \to t 1} \qquad ( 3 )$$

where Coefficient C is a function of at least one of device aerosolization power, aerosol chemical, and physical properties (e.g., aerosol particle size, aerosol particle surface charge, aerosol particle hydrophobicity, etc.). In some embodiments, the Coefficient C is a value in a range of approximately 0.001 to 0.80, inclusive. The constant C may be determined based at least in part on the test data or user data and adjusts the accumulated consumable consumption during the period t0 to t1 to account for inhalation losses, and inaccuracies.

In some embodiments, the controller 104 may be configured to receive and process individual operation data (e.g., one puff) and/or larger batches of data simultaneously. The estimated consumable emission per operation duration, as well as corresponding data, may be stored in a memory of the controller 104, and/or may be sent to the user device 108, the database(s) 140, and/or the base unit 120 of the aerosolization system 102. After estimating the consumable emission per operation duration, the controller 104 may be configured to generate a signal (e.g., a first signal) indicative of the estimated consumable emission per operation duration. The signal may include an analog signal, a digital signal, a real-time or delayed signal (e.g., data is logged and then the signal including the data may be generated), a wired or wireless signal and may include one or more of a continuous signal, a discrete signal, deterministic or non-deterministic signal, even or odd signal, a period or aperiodic signal, any other suitable signal or a combination thereof. Thus, the system 100 allows for estimation of consumable emissions that obviates the need to physically measure changes in consumable mass or volume over a period of time.

In some embodiments, the controller 104 may also be configured to generate a second signal (e.g., any of the signals disclosed herein) that may be indicative of the accumulated consumable consumption, and/or the consumable absorption in the human body (e.g., the user's body) during the time period t0 to t1, as previously disclosed herein. The second signal may include an analog signal, a digital signal, a real-time or delayed signal, a wired or wireless signal and may include one or more of a continuous signal, a discrete signal, deterministic or non-deterministic signal, even or odd signal, a period or aperiodic signal, any other suitable signal, or a combination thereof. In some embodiments, the controller 104 may be configured to generate one signal that includes information or data associated with each of the first signal and the second signal, as disclosed herein. In some embodiments, the first signal and thee second signal may be received and displayed on an output device (e.g., screen, display, user-interface, etc.).

In particular embodiments, the signal (e.g., the first signal and/or the second signal) may include a real-time signal. Generating real-time signals may provide the advantage of allowing a user to monitor in real time, the amount of consumable aerosol or vapor being generated by the aerosolization system 102, consumption of the consumable aerosol or vapor, and/or the amount of consumable being absorbed in the user's body over a period of time, as disclosed herein. In some embodiments, the first and/or second signal may be communicated to the user (e.g., to the user device 108) and/or a caregiver (e.g., a medical provider system, doctor, family member, friend, etc.) allowing the user and/or caregiver to receive real-time feedback, thus enabling real-time consumable consumption tracking and monitoring capabilities, which can lead to a specific, measurable, and attainable time-bound of control for consumable consumption and/or cessation. Such real-time feedback may have a significant impact on the user's behavior, for example, it may facilitate setting up and adhering to dosage limits, and/or reduce consumption of consumable based at least in part on an indication of how much consumable has been absorbed in the user's body.

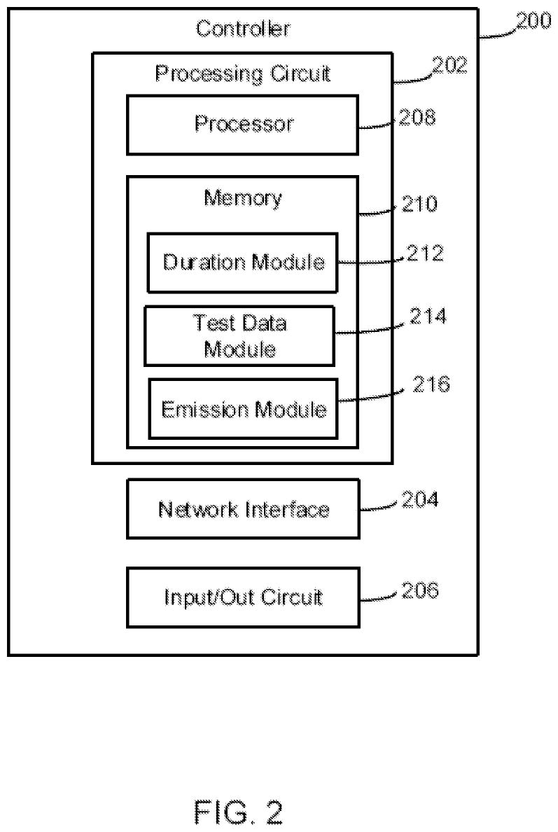

FIG. 2 is a schematic block diagram of a controller 200, according to an embodiment. The controller 200 may be functionally and/or structurally similar to the controller 104 of FIG. 1. The controller 200 may be configured to estimate consumable emissions for an aerosolization system (e.g., functionally and/or structurally similar to the aerosolization device 102). The controller 200 may include a processing circuit 202, a network interface 204, and an input/output circuit 206.

The processing circuit 202 may include a processor 208 and a memory 212. The processor 212 may be implemented as a general-purpose processor, an Application Specific Integrated Circuit (ASIC), one or more Field Programmable Gate Arrays (FPGAs), a Digital Signal Processor (DSP), a group of processing components, or other suitable electronic processing components. The memory 210 (e.g., Random Access Memory (RAM), Read-Only Memory (ROM), Non-volatile RAM (NVRAM), Flash Memory, hard disk storage, etc.) may store data and/or computer code for facilitating at least some of the various processes disclosed herein. The memory 210 may include tangible, non-transient volatile memory, or non-volatile memory.

The memory 212 may include a duration module 212, a test data module 214, and an emission module 214. The duration module 212 may store data and/or instructions for facilitating determination of a duration of an operation. For example, the duration module 212 may be configured to receive operation data (e.g., sensors data, input data, etc.) and may comprise instructions configured to determine an operation duration based at least in part on the operation data.

The test data module 214 may include data and/or instructions for handling test data of a test aerosolization system. The test data module 214 may store test data, generate test data, and/or process test data. For example, if the controller 200 receives processed test data, the test data module 214 may store the test data and send the test data when prompted. If the controller 200 is involved in the testing of a test aerosolization system, the test data module 214 may generate test data from the testing process and may process the data so that it may be used in other calculations. The emission module 214 may include data and/or instructions for estimating a consumable emission per operation. The emission module 214 may be configured to pull or otherwise receive data from the test data module 214 and the emission module 216 that is desired for estimating a consumable emission per operation. The emission module 214 may store equations used to estimate the consumable emission per operation such as equation (1).

The network interface 204 may be configured to send and/or receive data over the communication network 110 (e.g., to and from the user device 108, etc.). Accordingly, the network interface 204 may include any of a cellular transceiver (for cellular standards), local wireless network transceiver (for 802.11X, ZigBee, Bluetooth, Wi-Fi, or the like), wired network interface, a combination thereof (e.g., both a cellular transceiver and a Bluetooth transceiver), and/or the like. The input/output circuit 206 may be configured to facilitate sending and receiving data, signals, information, and the like. The input/output circuit 206 may include a port (e.g., serial port, USB port, etc.) that allows for the transfer of data and/or power into and/or out of the controller 200. The input/out circuit 206 may be configured to communicatively couple to at least one of a base unit of an aerosolization system (such as the aerosolization system 102) and a user device (such as the user device 108).

### User Device

As also mentioned above, the emission system may 100 further comprise a user device 108 communicably coupled to the aerosolization system 102. The user 106 may operate the user device 108 to communicate with the aerosolization system 102 and/or the controller 104. The user device 108 may allow for a user 106 to see, in real-time, information pertaining to aerosolization system 102 operation. For example, the user device 108 may display the consumable emission per operation duration as calculated by the controller 104. In some embodiments, the user device 108 may display a summary of aerosolization system information. The user device 108 may include, for example a mobile phone (e.g., an iPHONE®, an ANDROID® phone, a WINDOWS® phone, a SYMBIAN® phone or the like), a tablet computer, a personal computer (e.g., a desktop or a laptop), a smart TV, a smart watch, a gaming system, an IP TV box, or any other user device. The user device 108 may be configured to send and/or receive signals from the controller 104 and/or the base unit 130 of the aerosolization system 102. In some embodiments, the controller 104 may be integrated in the user device 108, or the instructions stored in a memory of the controller 104 may instead be stored in a memory of the user device 108 and configured to be executed by a processor of the user device 108.

### Communication Network

The user device 108, the controller 104, the database(s) 140, and the base unit 130 of the aerosolization system may be communicably coupled to a communication network 110. The communication network 110 may be structured to permit the exchange of data, values, instructions, messages, and the like between the base unit 130 of the aerosolization system 102, the controller 104, the database(s) 140, and/or the user device 108. The communication network 110 may be any suitable Local Area Network (LAN) or Wide Area Network (WAN). For example, the communication network 110 may be supported by Frequency Division Multiple Access (FDMA), Time Division Multiple Access (TDMA), Code Division Multiple Access (CDMA) (particularly, Evolution-Data Optimized (EVDO)), Universal Mobile Telecommunications Systems (UMTS) (particularly, Time Division Synchronous CDMA (TD-SCDMA or TDS) Wideband Code Division Multiple Access (WCDMA), Long Term Evolution (LTE), evolved Multimedia Broadcast Multicast Services (eMBMS), High-Speed Downlink Packet Access (HSDPA), and the like), Universal Terrestrial Radio Access (UTRA), Global System for Mobile Communications (GSM), Code Division Multiple Access 1× Radio Transmission Technology (1×), General Packet Radio Service (GPRS), Personal Communications Service (PCS), 802.11X, ZigBee, Bluetooth, Wi-Fi, any suitable wired network, combination thereof, and/or the like.

### Methods

Methods for estimating a consumable emission per user operation duration of an aerosolization system (e.g., the aerosolization system 100) may generally comprise receiving consumable emission test data of a test aerosolization system based at least in part on a test aerosolizer power or a range of a test aerosolizer powers of a test aerosolizer of the test aerosolization system and a test consumable concentration or a range of test consumable concentrations. The methods may also comprise receiving operation data of a user aerosolization system corresponding to an operation duration based at least in part on an input signal from a user, the operation data comprising at least a consumable concentration of a consumable included in the user aerosolization system and an aerosolizer power level of an aerosolizer of the user aerosolization system during the operation duration. The methods may also comprise estimating a consumable emission per operation duration based at least in part on the operation data and consumable emission test data, and generating, by the controller, a signal indicative of the estimated consumable emission per operation duration.

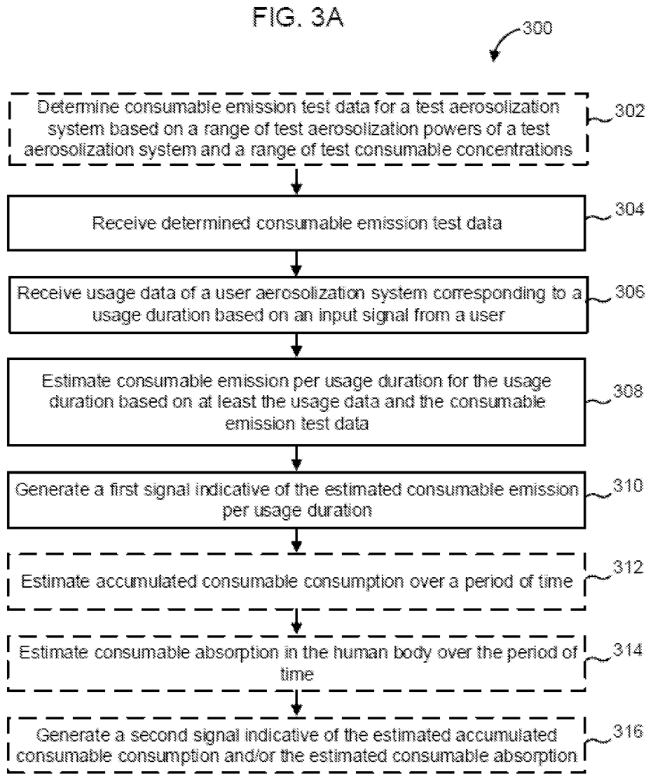

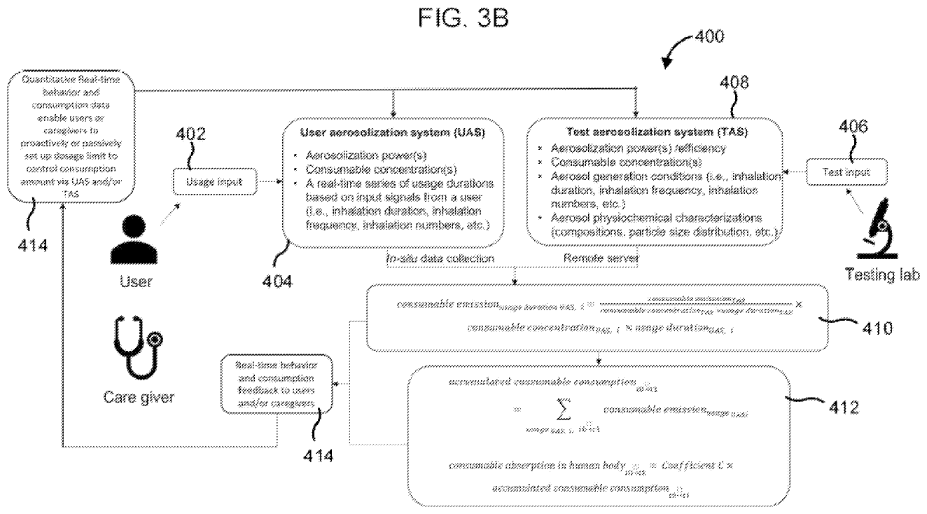

For example, FIG. 3A depicts a flow chart of a method 300 for estimating a consumable emission per operation duration, according to an embodiment. While described with respect to the controller 104, it should be appreciated that the operations of the method 300 may performed by any other suitable controller or control unit capable of performing the operations of the method 300, as disclosed herein.

The method 300 may include determining consumable emission test data for a test aerosolization system based at least in part on a range of test aerosolizer powers of an aerosolizer of the test system and a range of test consumable concentrations. The consumable emission test data may be determined by a device, such as a controller (e.g., the controller 104), in an emission system, such as emission system 100, or may be determined by an outside system and stored in a database, such as the database(s) 140. The range of test aerosolization powers may include values of power outputs or may be generic power values (e.g., low, medium, high, etc.). The range of test consumable concentrations may be values of consumable concentrations (e.g., in a range of about 3 mg/ml to about 12 mg/ml, inclusive) or may be generic consumable concentrations (e.g., low, medium, high, etc.). The determined consumable emission test data corresponds to the specific test aerosolizer powers and range of test consumable concentrations. In some embodiments, operation 302 may be optional.

At 304, the estimating a consumable emission per operation may further comprise receiving determined consumable emission test data at the controller 104. The consumable emission test data may be additionally processed by the controller 104 to generate test data that is suitable for further operations. At 306, the method 300 may comprise receiving operation data of a user aerosolization system by the controller 104. The operation data may correspond to an operation duration based at least in part on an input signal from a user. The operation data may be additionally processed to determine an operation duration. For example, the operation data may include the amount of time an activation mechanism is actuated. For example, in some variations, the operation data may include the amount of time a switch (e.g., a switch 141) is actuated (e.g., pressed down) on the user aerosolization system 102 (or any other user aerosolization system), or a draw pressure is exerted on a draw sensor (e.g., the draw sensor 143) of the user aerosolization system (e.g., the aerosolization system 102). To determine the operation duration, the amount of time may be modified to account for an aerosolizer (e.g., the aerosolizer 127 or 137) energizing (e.g., heat-up) time.

At 308, the method 300 may further comprise estimating, using the controller 104, consumable emission per operation duration for the operation duration based at least in part on at least the operation data and the consumable emission test data. In some embodiments, the consumable emission per operation duration may be based at least in part on a test consumable emission of the consumable emission test data, a test consumable concentration, and a test operation duration of the emission test data. In some embodiments, the consumable emissions per operation duration may be based at least in part on the consumable concentration and the operation duration. In some embodiments, methods may further comprise estimating consumable emission per operation duration using equation (1). In some embodiments, methods may further comprise storing the estimated consumable emission per operation duration in a memory for future access. In some embodiments, methods may further comprise determining a total consumable emission. In some variations, determining a total consumable emission may comprise summing a plurality of consumable emission per operation duration.

At 310, the method 300 may further comprise generating, using the controller 104, a signal indicative of the estimated consumable emission per operation duration. The signal may be sent to the user device 108 to display the estimated consumable emission per operation duration. In some embodiments, the signal may be to a memory or database, such as the database 140, configured to store the consumable emission per operation duration data where it may be accessed. In some embodiments, operation 310 may be optional.

At 312, the method 300 may further comprise estimating, using the controller 104, an accumulated consumable consumption over a period of time. The accumulated consumable consumption represents the amount of consumable that may be consumed by the user if all the aerosol or vapor generated by the aerosolization system 102, between a first time period and a second time period, were absorbed. In some embodiments, the accumulated consumable consumption is based at least in part on consumable consumption per operation duration, operation number, operation frequency, etc. In some embodiments, the methods may further comprise estimating accumulated consumable consumption over a period of time using equation (2). In some embodiments, operation 312 may be optional.

At 314, the method 300 may further comprise estimating, using the controller 104, consumable absorption in the human body over the period of time. The total amount of vapor generated by the aerosolization system 102 may not be the actual amount consumed by the user. For example, the user may exhale some amount of the consumable while inhaling, or some amount of consumable aerosol may be lost during inhalation because of, for example, a non-hermetic seal between the user's mouth and a mouthpiece of the cartridge 120. The actual amount of consumable absorbed in the body of the user may be less than the amount of aerosol inhaled by the user. In some embodiments, the method may further comprise estimating consumable absorption in the human body over a period of time using equation (3), where C is a constant. In some embodiments, C is a value in the range of 0.001 to 0.80. In some embodiments, the method may further comprise, determining C based at least in part on test data and/or user data and weighting the accumulated consumable consumption during the time period to account for inhalation losses, and inaccuracies. In some embodiments, operation 314 may be optional.

At 316, the method 300 may further comprise generating a second signal indicative of the estimated accumulated consumable consumption and/or the estimated consumable absorption. The signal may be sent to the user device 108 to display the estimated accumulated consumable consumption and/or estimated consumable absorption. In some embodiments, the signal may be to a memory or database, such as the database 140, configured to store the estimated accumulated consumable consumption and/or the estimated consumable absorption where it may be accessed. In some embodiments, operation 316 may be optional.

FIG. 3B is a schematic flow chart of another method 400 for estimating consumable emission as well as estimating total consumable emission over a period of time and amount of consumable absorbed in a user's body over the period of time, according to an embodiment.

The method 400 may include receiving, by a user aerosolization system, an operation input at 402. The method 400 further may include, collecting, by an in-situ data collection system, information from the user aerosolization system at 404. The information may correspond to the operation input and/or the aerosolization system, such aerosolization power(s), consumable concentration(s), real-time series of operation durations based at least in part on input signals from a user (e.g., inhalation duration, inhalation frequency, inhalation numbers, etc.), and/or the other information regarding the use aerosolization system and/or the operation input.

At 406, the method 400 may include receiving, by a test aerosolization system, a test input. At 408, the method 400 may include receiving, by a remote server, information from the test aerosolization system at 408. The information may include aerosolization power(s), aerosolization efficiency, consumable concentration(s), aerosol generation conditions (e.g., inhalation duration, inhalation frequency, inhalation numbers, etc.), and/or aerosol physiochemical characterizations (e.g., compositions, particle size distribution, etc.), and/or other information regarding the test aerosolization system and/or the test input.

At 410, the method 400 may include determining a consumable emission per operation duration for the user aerosolization system based at least in part on information received from the in-situ data collection and the remote server. In some embodiments, the method 400 may include determining a consumable emission per operation duration using equation (1). In some embodiments, determining a consumable emission per operation duration may be based at least in part on a consumable emission, a consumable concentration, and an operation duration from the test aerosolization system, and a consumable concentration and an operation duration of the user aerosolization system.

At 412, the method 400 may include determining at least one of an accumulated consumable consumption and a consumable absorption in the human body based at least in part on the consumable emission user operation duration. In some embodiments, the method 400 may include determining the accumulated consumable consumption using equation (2). In some embodiments, the method 400 may include determining the consumable absorption in human body using equation (3).

At 414, the method 400 may include generating real-time behavior and consumption feedback and providing the feedback to users and/or caregivers based at least in part on at least one of the consumable emission per operation duration, the accumulated consumable consumption, and the consumable absorption in human body. The quantitative real-time behavior and consumption feedback and data enables users and/or caregivers to proactively or passively set up dosage limits to control consumption amount via the user aerosolization system and/or the test aerosolization system. Once the users and/or the caregivers review the real-time behavior and consumption feedback, the method 400 may restart using the feedback as an input.

### Experimental Examples

The following section describes experimental examples associated with consumable emission test data collection and estimating consumable emission per operation duration based at least in part on an operation data and consumable emission test data. These examples are merely illustrations and should not be construed as limiting the disclosure.

### Nicotine Consumption During Ad-Libitum Use

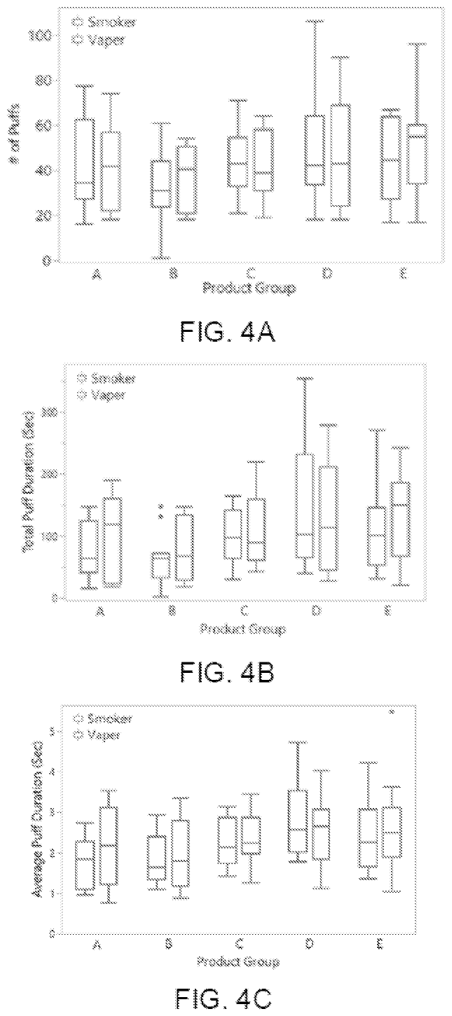

Referring generally to FIGS. 4-9, the feasibility of estimating nicotine consumption during ad-libitum use of a puff recording electronic nicotine delivery system (PR-ENDS) device is discussed. It should be appreciated that while the method and embodiments disclosed herein in reference to FIGS. 4-9 relate to nicotine and nicotine-use, these methods may be applied to other consumables. To establish the feasibility of using the consumable emission estimation methods disclosed herein, a one-way randomized, controlled, open-label puff topography (e.g., representation how aerosolization system characteristics and user behavior impact consumable such as nicotine emission) and nicotine pharmacokinetic (PK) assessment was carried out in 24 healthy adult smokers and vapes. Participants were assigned with randomized product use sequence of PR-ENDS during both controlled and ad-libitum use sessions. During the ad-libitum use session, puff topography was measured by a clinical research support system (CReSS), which represents test puff topography data associated with PR-ENDS, as a benchmark, as well as by PR-ENDS itself.

No significant differences of puff topography parameters (e.g., number of puffs, total puff duration, average puff duration) between PR-ENDS and CReSS were observed at population level and across different device powers, e-liquid nicotine strengths, and flavors. The evaluation of nicotine consumption estimated by PR-ENDS suggested that this device can be employed as a convenient tool for estimating a nicotine use without measuring e-liquid weight loss between puffs.

The PR-ENDS device used is an open, refillable ENDS product with a removable 0.8-ohm aerosolizer coil, 2 mL e-liquid capacity, and three power setting (low power: 7-9 watts, medium power 9-11 watts, and high power, 11-13 watts). The PR-ENDS device is able to measure puff data including number of puffs, puff durations, and puff intervals through a built-in mechanism and via securely connected Bluetooth. The puff duration is measured and recorded based at least in part on the time of pressing and holding the power button. The investigated PR-ENDS devices include two power settings (high and low), two nicotine strengths (12 mg/mL and 3 mg/mL) and two flavors (tobacco and menthol).

TABLE 1
Description of five investigated PR-ENDS groups (A-E)

| Product | | | | |
|---|---|---|---|---|
| Group | Flavor | Nicotine Strength | Power | Figure and Table Code |
| A | Tobacco | 12 mg/mL | High | A (Tobacco/12/High) |
| B | Menthol | 12 mg/mL | High | B (Tobacco/12/High) |
| C | Tobacco | 12 mg/mL | Low | C (Tobacco/12/Low) |
| D | Tobacco | 3 mg/mL | Low | D (Tobacco/3/Low) |
| E | Tobacco | 3 mg/mL | High | E (Tobacco/3/High) |

Potential participants were examined for eligibility of the study during an initial screening operation. The individuals were included if they (1) were healthy males or females within the ages of 21 to 65 years, (2) were either current smokers (≥10 per day) of factory-made combustible cigarettes (eCO>10 ppm at screening) for at least 3 continuous months and may be occasional users of e-cigarette, but none in the 14 days before the screening visit; or current daily users of e-cigarette devices with an e-liquid nicotine concentration >0 mg/mL (eCO≤10 ppm at screening) for at least 3 continuous months and may be occasional users of combustible cigarettes, but none in the 14 days before the screening visit; (3) had urine cotinine >200 ng/mL at screening.

Following the screening operation, participants attended the study site six times including one visit using their own usual brand combustible cigarettes (smoker) or e-cigarettes (vaper), and five visits using PR-ENDS device pre-filled with assigned freebase nicotine e-liquids in a randomized product use sequence (details shown in Table 1). Participants were provided with a supply of their assigned PR-ENDS product to use at home before their next visit for familiarization purposes. Each individual visit was separated by a minimum of 24-hour washout period. During visits, each participant had two use sessions. In the first controlled use session, participants started by taking 10 puffs, 30 seconds apart, followed by a period of 120 min to allow nicotine plasma concentration to ramp down to baseline. In the second ad libitum use session, participants were allowed to take as many puffs as desired during a period of 60 minutes. Throughout both controlled use session and ad libitum use session, blood samples were obtained for plasma nicotine analysis and questionnaires were administered at various time points. During the 1-hour ad libitum use session, e-liquid weight loss for assessment of nicotine consumption was calculated based at least in part on the weight difference before and after use sessions. Nicotine plasma concentration at 0 min, 30 min, and 60 min of 1-hour use was obtained based at least in part on nicotine analysis from blood samples. The plasma concentration-time curve (AUC_{ad lib}) was calculated based at least in part on the time course of measured nicotine concentration from 0 min to 60 min. The puff topography parameters including number of puffs, total puff duration, and average puff duration were measured by a CReSS device attached to PR-ENDS as the benchmark.

Descriptive statistics including means and standard deviation (SD) were calculated for each variable. The box plots of targeted variables were presented in figures. Two-sample t-tests and paired t-tests were applied to identify any statistically significant difference between compared samples. Variables were deemed significant at the level of 0.05 (α=0.05). A one-way or repeated measures analysis of variables (ANOVA) and Dunnett's multiple comparison tests were conducted to assess stratified differences between product use groups (A-E). Correlation coefficient (R²) was further calculated to assess the linear relations between compared variables.

As shown in Table 2, the number of puffs showed average values of 33.3-49.5 puffs for group A-E in smokers and 36.5-49.7 puffs in vapers. Total puff duration showed average values of 63.3-146.6 seconds for group A-E in smokers and 77.1-133.4 seconds in vapers. Average puff duration showed average values of 1.77-2.83 seconds for group A-E in smokers and 1.97-2.60 seconds in vapers. The box plots of puff topography parameters measured by PR-ENDS are shown in FIGS. 4A-4C.

TABLE 2
Summary of puff topography parameters measured by PR-ENDS

| | | | Total puff | Average puff |
|---|---|---|---|---|
| | Product | Number of | duration | duration |
| | Group | Puffs | (second) | (second) |
| Smoker | A (Tobacco/12/ | 41.8 (20.2) | 76.9 (43.7) | 1.77 (0.59) |
| | High) | | | |
| | B (Menthol/12/ | 33.3 (17.6) | 63.3 (40.4) | 1.84 (0.61) |
| | High) | | | |
| | C (Tobacco/12/ | 44.5 (14.3) | 101.3 (41.6) | 2.23 (0.60) |
| | Low) | | | |
| | D (Tobacco/3/ | 49.5 (25.3) | 146.6 (95.4) | 2.83 (1.01) |
| | Low) | | | |
| | E (Tobacco/12/ | 45.4 (17.7) | 113.2 (66.1) | 2.39 (0.84) |
| | High) | | | |
| Vaper | A (Tobacco/12/ | 42.3 (18.7) | 101.0 (62.6) | 2.15 (0.97) |
| | High) | | | |
| | B (Menthol/12/ | 36.5 (13.7) | 77.1 (46.8) | 1.97 (0.85) |
| | High) | | | |
| | C (Tobacco/12/ | 42.1 (14.4) | 103.4 (53.8) | 2.36 (0.62) |
| | Low) | | | |
| | D (Tobacco/3/ | 48.4 (24.0) | 133.4 (87.9) | 2.52 (0.84) |
| | Low) | | | |
| | E (Tobacco/12 | 49.7 (21.9) | 133.4 (72.3) | 2.60 (1.22) |
| | /High) | | | |

For the smoker group, PR-ENDS recorded data showed that higher device power (e.g., A vs. C and E vs. D) and nicotine strength (e.g., A vs. E and C vs. D) yielded lower values of number of puffs, total puff duration, and average puff duration. Yet, a dissimilar trend was observed for the vaper groups in that the power and nicotine strength may have limited effect on puff topography parameters. Such difference might potentially indicate different puffing behaviors between smokers and vapers. For both smoker and vaper groups, PR-ENDS prefilled with menthol flavored e-liquid yielded lower # of puffs and lower total puff duration comparted with tobacco flavored e-liquid (A vs. B). However, such difference was not statistically significant (p>0.05 based at least in part on ANOVA) given the large variation of actual puff behavioral data in both smokers and vapers.

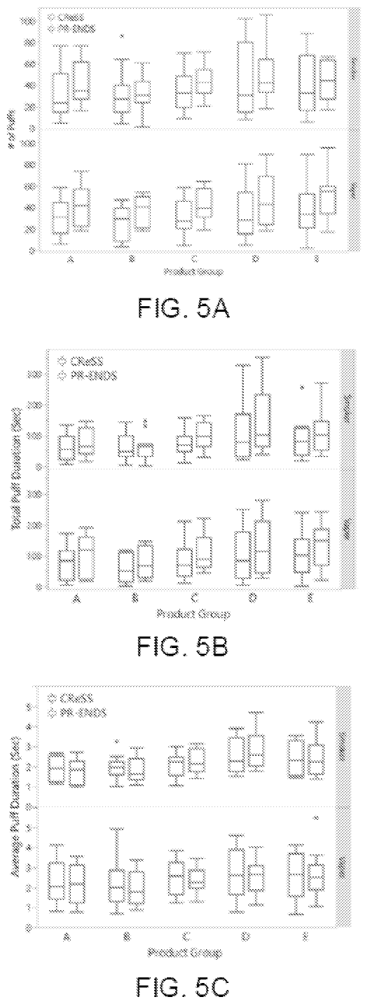

The observed puff topography parameters measured by PR-ENDS were verified through the use of a CReSS device to serve as the benchmark puff sensor to compare with PR-ENDS in recorded puff data. CReSS, however, has a low sensitivity for low puff flow rate which may affect results when assessing puff numbers from CReSS. As seen in FIGS. 5A-5C, the box plots of puff topography parameters showed comparable values on number of puffs, total puff duration, and average puff duration between CReSS and PR-ENDS among smokers and vapers. Based at least in part on the puff topography measured by CReSS (shown in Table 3), higher PR-ENDS device power and higher e-liquid nicotine strength were associated with lower values of number of puffs, total puff duration, and average puff duration in smoker group, which is consistent with the observation made by PR-ENDS. For the vaper group, the correlation between device power/nicotine strength and puff parameters is less evident, which also concurred with findings discovered by PR-ENDS as mentioned above.

TABLE 3
Summary of puff topography parameters measured by CReSS

| | | | Total | Average |
|---|---|---|---|---|
| | | Number | puff | puff |
| | Product | of | duration | duration |
| | Group | Puffs | (second) | (second) |
| Smoker | A (Tobacco/ | 31.8 (22.2) | 60.3 (41.6) | 1.88 (0.60) |
| | 12/High) | | | |
| | B (Menthol/ | 30.9 (23.5) | 62.1 (45.2) | 1.97 (0.58) |
| | 12/High) | | | |
| | C (Tobacco/ | 38.1 (18.9) | 77.8 (39.5) | 2.05 (0.57) |
| | 12/Low) | | | |
| | D (Tobacco/ | 44.9 (34.8) | 106.9 (91.1) | 2.52 (0.88) |
| | 3/Low) | | | |
| | E (Tobacco/ | 40.0 (26.2) | 89.5 (68.2) | 2.39 (0.79) |
| | 12/High) | | | |
| Vaper | A (Tobacco/ | 30.5 (18.2) | 80.5 (60.2) | 2.31 (1.19) |
| | 12/High) | | | |
| | B (Menthol/ | 25.0 (15.0) | 61.0 (44.9) | 2.27 (1.22) |
| | 12/High) | | | |
| | C (Tobacco/ | 31.4 (16.9) | 77.7 (60.0) | 2.38 (0.85) |
| | 12/Low) | | | |
| | D (Tobacco/ | 34.8 (24.4) | 101.8 (88.2) | 2.52 (1.17) |
| | 3/Low) | | | |
| | E (Tobacco/ | 38.9 (21.4) | 110.7 (72.8) | 2.58 (1.04) |
| | 12/High) | | | |

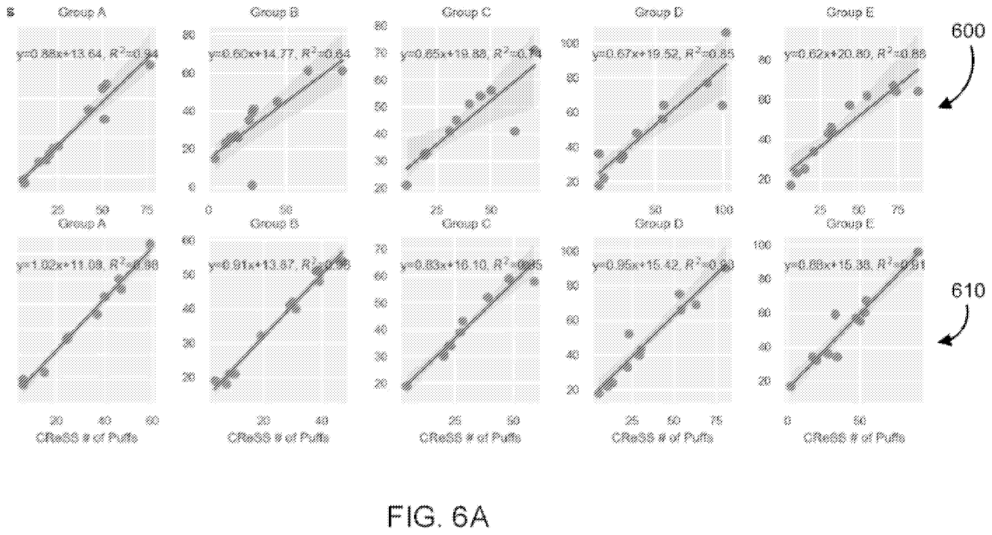

Statistical comparison tests were conducted in order to assess the comparability of puff topography parameters measured between CReSS and PR-ENDS. The results in Table 4 show that no statistically significant differences were identified based at least in part on p-values of two-sample t-test comparisons, which indicated an agreement of puff topographies measured between PR-ENDS and CReSS at a population level. The results in table 4 further show it may be feasible to use PR-ENDS as a non-interventional platform to assess users' naturalistic puff topography, yielding a same level of puff recording sensitivity and accuracy as CReSS. Linear correlation analysis of puff topography parameters between CReSS and PR-ENDS further showed that high correlation coefficients (R²) exist between data recorded by CReSS and PR-END, as seen in FIGS. 6A-6C.

TABLE 4
Two Sample t-test of puff topography
parameters between CReSS and PR-ENDS

| | | p-value | | |
|---|---|---|---|---|
| | | | Total | Average |
| | | Number | puff | puff |
| | Product | of | duration | duration |
| | Group | Puffs | (second) | (second) |
| Smoker | A (Tobacco/12/High) | 0.265 | 0.360 | 0.644 |
| | B (Menthol/12/High) | 0.778 | 0.945 | 0.600 |
| | C (Tobacco/12/Low) | 0.405 | 0.224 | 0.518 |
| | D (Tobacco/3/Low) | 0.716 | 0.318 | 0.427 |
| | E (Tobacco/12/High) | 0.566 | 0.397 | 0.986 |
| Vaper | A (Tobacco/12/High) | 0.170 | 0.476 | 0.742 |
| | B (Menthol/12/High) | 0.063 | 0.410 | 0.498 |
| | C (Tobacco/12/Low) | 0.124 | 0.313 | .946 |
| | D (Tobacco/3/Low) | 0.204 | 0.421 | .999 |
| | E (Tobacco/12/High) | 0.279 | 0.498 | 0.967 |

Puff topography measurement provides a quantifiable base to estimate the amount of consumed nicotine. In theory, nicotine emission measured under certain puff topography, combined with the number of puffs and puff duration in situ, can estimate how much of nicotine gets aerosolized during user's inhalation process. On the other hand, nicotine consumption, defined as the amount of nicotine contained in e-liquid consumed by users, is an important factor in puff topography assessment. Nicotine consumption directly represents users' nicotine use, addiction, and abuse liability, especially when it is measured in an uncontrolled environment (e.g., ad libitum use).

In order to determine if lower device power and lower nicotine strength essentially lead to a lower nicotine consumption, nicotine consumption during the ad libitum use of PR-ENDS prefilled with designated e-liquids (group A-E) was estimated. Specifically, it was calculated by integrating PR-ENDS measured puff topography data and laboratory tested nicotine emission results into equation (2).

$$Nicotine consumption = \frac{Nicotine emission}{Testing puff duration} ⨯ Total puff duration \qquad ( 2 )$$

Nicotine consumption refers to the amount of nicotine consumed by PR-ENDS users during the ad libitum use session. Nicotine emission values (as seen in Table 5) was obtained from laboratory testing with a puff regime of 3 seconds as the testing puff duration. In group A-E, the nicotine emission had different values, as they were measured using different device power (High or Low), e-liquid nicotine strength (12 mg/mL or 3 mg/mL) and flavors (tobacco or menthol). The same setup of PR-ENDS device was utilized in laboratory testing to guarantee the reproducibility of nicotine emission results. Total puff duration was obtained by either summing puff duration of each individual puff recorded by PR-ENDS or directly read from PR-ENDS puff recording coil chip.

TABLE 5
Summary of average nicotine emission per puff for PR-ENDS

| | | Nicotine | Average |
|---|---|---|---|
| | | concentration | nicotine |
| | Puff | of e-liquid | emission |
| Device power | duration | (mg/ml) | (mg/puff) |
| High | 3 seconds | 3 | 0.0180 |
| | 3 seconds | 12 | 0.0952 |
| Medium | 3 seconds | 3 | 0.0144 |
| | 3 seconds | 12 | 0.0762 |
| Low | 3 seconds | 3 | 0.0111 |
| | 3 seconds | 12 | 0.0406 |

Nicotine consumption can then be calculated to represent how much nicotine has been consumed during the ad libitum use session. The assumption applied in the calculation is that nicotine consumption is projected as linearly proportional to the puff duration measured by PR-ENDS, which means that there is no need to involve puff volume or puff flow rate (puff volume over unit puff duration) into the calculation. This assumption is applicable as puff flow rate or puff volume do not impact aerosol emission yield, and that puff duration alone is sufficiently representative for estimating the aerosolized nicotine generated from e-cigarettes.

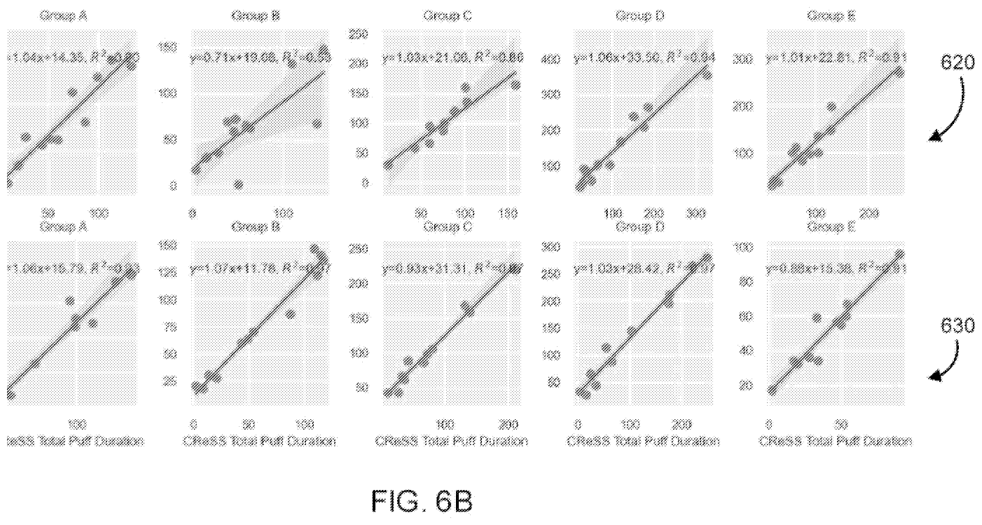

The emission value was alternatively obtained by calculating e-liquid weight loss to validate the assumption that nicotine consumption can be estimated based at least in part on PR-ENDS recorded puff topography data and laboratory tested aerosol emissions. FIG. 7 depicts the compared box plots of nicotine consumption between PR-ENDS method and e-liquid weight loss method demonstrating that the two approaches yielded comparable values of nicotine consumption across groups A-E in smokers and vapers. The PR-ENDS derived nicotine consumption shoed average values of 0.48-2.40 mg for group A-E in smokers and 0.40-2.63 mg in vapers; e-liquid weight loss derived nicotine consumption showed average values of 0.58-2.00 mg for group A-E smokers and 0.56-2.96 mg in vapers (as seen in Table 6).

TABLE 6
Summary of nicotine consumption estimated by (1) puff topography
parameters measured by PR-ENDS and (2) e-liquid weight loss

| | | PR-ENDS | e-liquid Weight |
|---|---|---|---|
| | | Derived | Loss Derived |
| | | Nicotine | Nicotine |
| | Product | Consumption | Consumption |
| | Group | (mg) | (mg) |
| Smoker | A (Tobacco/12/High) | 2.40 (1.42) | 2.00 (1.72) |
| | B (Menthol/12/High) | 1.98 (1.32) | 1.76 (1.40) |
| | C (Tobacco/12/Low) | 1.11 (0.74) | 1.42 (0.91) |
| | D (Tobacco/3/Low) | 0.48 (0.33) | 0.58 (0.55) |
| | E (Tobacco/3/High) | 0.88 (0.54) | 0.83 (0.68) |
| Vaper | A (Tobacco/12/High) | 2.63 (2.23) | 2.96 (2.36) |
| | B (Menthol/12/High) | 2.41 (1.53) | 2.25 (1.80) |
| | C (Tobacco/12/Low) | 1.25 (0.81) | 1.62 (1.16) |
| | D (Tobacco/3/Low) | 0.40 (0.32) | 0.56 (0.47) |
| | E (Tobacco/3/High) | 0.95 (0.64) | 1.05 (0.84) |

In Table 7, two-sample t-tests and paired t-tests of nicotine consumption between PR-ENDS and e-liquid weight loss methods showed that no statistically significant differences were identified.

TABLE 7
Statistical comparison of nicotine consumption between value estimated
based at least in part on puff topography measured by PR-ENDS and
value estimated based at least in part on e-liquid weight loss

| | | Two sample | Paired |
|---|---|---|---|
| | Product Group | t-test p-value | t-test p-value |
| Smoker | A (Tobacco/12/High) | 0.511 | 0.454 |
| | B (Menthol/12/High) | 0.683 | 0.645 |
| | C (Tobacco/12/Low) | 0.336 | 0.363 |
| | D (Tobacco/3/Low) | 0.563 | 0.441 |
| | E (Tobacco/3/High) | 0.816 | 0.700 |
| Vaper | A (Tobacco/12/High) | 0.703 | 0.522 |
| | B (Menthol/12/High) | 0.808 | 0.666 |
| | C (Tobacco/12/Low) | 0.334 | 0.066 |
| | D (Tobacco/3/Low) | 0.324 | 0.070 |
| | E (Tobacco/3/High) | 0.784 | 0.543 |

To further validate that nicotine consumption can be estimated based at least in part on PR-ENDS recorded puff topography data and laboratory tested aerosol emissions, a repeated-measures analysis of variance (ANOVA) determines if there are statistical differences in nicotine consumption across five PR-ENDS product groups (A-E). The results of the repeated-measures ANOVA showed that for both smokers (p<0.0001) and vapers (p=0.0002), the nicotine consumptions were significantly different among groups A-E. Table 8 shows a Dunnett's multiple comparisons test (control: group A Tobacco/12/High) that shows that the device power (e.g., A vs. C) and nicotine strength (e.g., A vs. E) impacted nicotine consumption. More specifically, for both smoker and vaper groups, higher device power (e.g., A vs. C and E vs. D) and nicotine strength (e.g., A vs. E and C vs. D) led to higher nicotine consumptions during PR-ENDS use, even though users may freely puff titrate the device (e.g., puff more times or longer puffs) with lower device power and lower nicotine strengths. As such, PR-ENDS demonstrated that by limiting device power and nicotine strengths, both can effectively reduce the nicotine consumption. This conclusion may be reached based at least in part on the recorded puff topography data and laboratory tested nicotine emission results. Thus, there is no need to physically measure the e-liquid weight loss to estimate the nicotine use.

TABLE 8
Dunnett's multiple comparison test of nicotine consumption
(estimated by puff topography parameters measured
by PR-ENDS device) among product groups A-E

| | Product | | | |
|---|---|---|---|---|
| | Comparison | Nicotine | Nicotine | |
| | to A | Consumption | Consumption | |
| | (Tobacco/ | Mean | 95% Confidence | |
| | 12/High) | Difference | Interval | p-value |
| Smoker | B (Menthol/ | −0.4248 | −1.4207, 0.5710 | 0.662 |
| | 12/High) | | | |
| | C (Tobacco/ | −1.2862 | −2.2820, −0.2903 | 0.0072 |
| | 12/Low) | | | |
| | D (Tobacco/ | −1.5180 | −2.9125, −0.9208 | <0.0001 |
| | 3/Low) | | | |
| | E (Tobacco/ | −1.9167 | −2.5139, −0.5222 | 0.0012 |
| | 3/High) | | | |
| Vaper | B (Menthol/ | −0.2207 | −1.5569, 1.1154 | 0.982 |
| | 12/High) | | | |
| | C (Tobacco/ | −1.3746 | −2.7107, −0.0384 | 0.042 |
| | 12/Low) | | | |
| | D (Tobacco/ | −1.6725 | −3.5588, −0.8865 | 0.0004 |
| | 3/Low) | | | |
| | E (Tobacco/ | −2.2226 | −3.0087, −0.3364 | 0.0096 |
| | 3/High) | | | |

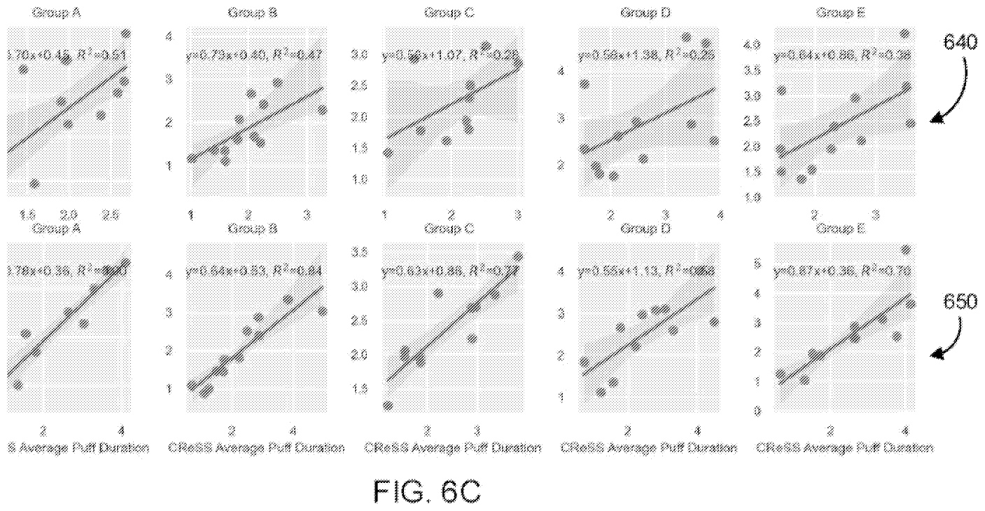

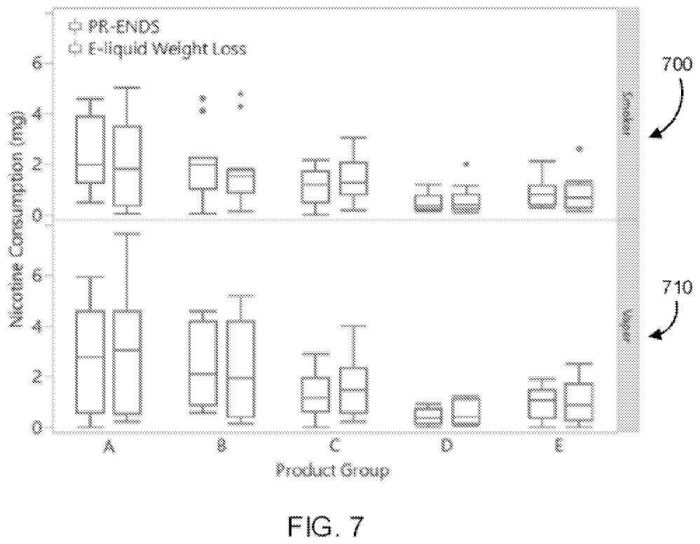

Plasma nicotine concentration (blood samples at 0 min, 30 min, and 60 min) and AUC_{ad lib} during 1-hour ad libitum use were calculated as a clinical validation of nicotine consumptions derived from PR-ENDS, the results of which are shown in Table 9 and FIG. 8. AUC represents the accumulated concentrations of nicotine in blood samples over a certain period of time, which can be treated as a proxy of the intake of nicotine inhaled in the human body. A linear regression analysis model between average nicotine consumption and average PK parameter AUC_{ad lib} was conducted to determine if PR-ENDS derived nicotine consumption can be used to estimate nicotine intake. FIG. 9 depicts a nearly linear (R: 0.915-0.979) relationship between PR-ENDS derived nicotine consumption and AUC_{ad lib} was presented. A similar linear relationship can be identified between e-liquid weight loss derived nicotine consumption and AUC_{ad lib}. Thus, it is viable to utilized puff recording e-cigarettes, such as PR-ENDS, to directly assess the puff topography, nicotine consumption and intake in a natural use environment.

TABLE 9
Summary of nicotine PK parameters (nicotine
concentration and AUC_{ad lib})

| | Product | C_{0 min} | C_{30 min} | C_{60 min} | AUC_{ad lib} |
|---|---|---|---|---|---|
| | Group | (ng/mL) | (ng/mL) | (ng/mL) | (min * ng/mL) |
| Smoker | A (Tobacco/ | 3.59 | 6.41 | 8.35 | 186 |
| | 12/High) | (2.11) | (3.50) | (5.60) | (121) |
| | B (Menthol/ | 3.44 | 6.31 | 7.46 | 177 |
| | 12/High) | (2.32) | (3.99) | (4.98) | (155) |
| | C (Tobacco/ | 2.74 | 4.90 | 5.89 | 135 |
| | 12/Low) | (1.76) | (1.89) | (2.45) | (112) |
| | D (Tobacco/ | 2.02 | 3.02 | 3.39 | 77.8 |
| | 3/Low) | (1.32) | (1.62) | (1.97) | (76.1) |
| | E (Tobacco/ | 1.85 | 3.78 | 3.98 | 82.6 |
| | 3/High) | (1.44) | (2.69) | (2.49) | (58.1) |
| Vaper | A (Tobacco/ | 5.04 | 7.53 | 11.34 | 295 |
| | 12/High) | (6.47) | (7.45) | (9.83) | (302) |
| | B (Menthol/ | 3.90 | 7.04 | 9.35 | 279 |
| | 12/High) | (4.38) | (5.98) | (7.88) | (307) |
| | C (Tobacco/ | 4.12 | 6.80 | 8.34 | 194 |
| | 12/Low) | (6.15) | (6.72) | (6.91) | (129) |
| | D (Tobacco/ | 2.58 | 3.67 | 3.95 | 81.1 |
| | 3/Low) | (3.11) | (3.34) | (2.98) | (75.4) |
| | E (Tobacco/ | 3.36 | 4.99 | 6.12 | 107 |
| | 3/High) | (4.70) | (5.94) | (6.73) | (94.9) |

### Behavior Analysis of Puff Recording Electronic Nicotine Delivery System

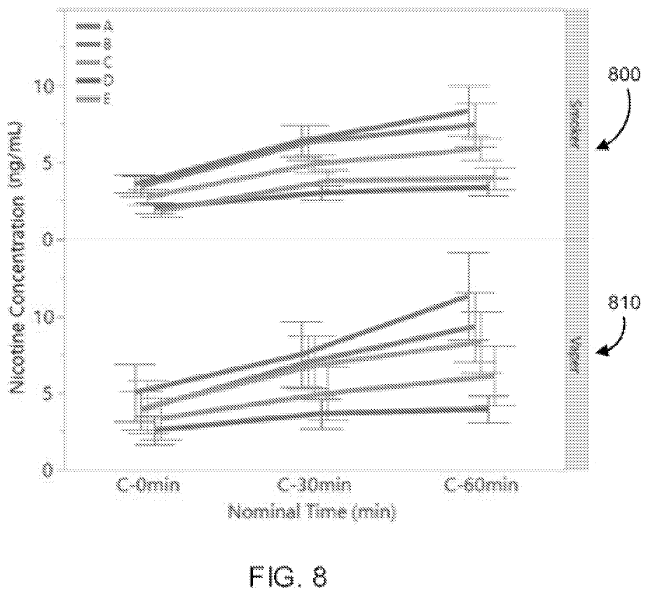

Referring generally to FIGS. 10A-16C, actual use behaviors of e-cigarettes with data collected from PR-ENDS device are shown. The methods discussed in reference to FIGS. 10A-16C are applied to nicotine and devices that are configured to generate aerosolized nicotine, however, it should be appreciated that other consumables may be used. The PR-ENDS device used is an open refillable device with a removable 0.8-ohm coil, 2 ml e-liquid capacity, and three power output settings (low power: 7-9 watts; medium power: 9-11 watts; and high power: 11-13 watts). The PR-ENDS device is able to measure puff parameters such as the number of puffs, puff duration, and puff intervals through a built-in chip in the device. The recorded data can then be uploaded to the cloud in real-time via a smartphone or computer-based app. PR-ENDS use information such as e-liquid nicotine concentration, e-liquid brands, device power, etc. can be simultaneously reported and obtained in situ and integrated with the recorded puff topography data to approximate device nicotine emission puff by puff.

The actual use behavior assessment of the PR-ENDS device consists of observing a single group of e-cigarette users enrolled in a product trial study. In total, 61 participants participated in the assessment. Each participant used the PR-ENDS device as their primary source of nicotine with their selected e-liquids for the trial duration. Additional inclusion criteria include: (1) older than 18 years; (2) does not have a history of chronic disease or psychiatric condition; (3) does not regularly use prescription medication; (4) not pregnant; and (5) not enrolled in a smoking cessation program. A baseline was conducted to collect information about participants' demographics and nicotine use history (cigarette and PR-ENDS).

After completing the baseline survey, participants used the PR-ENDS device in real-world condition for three weeks (e.g., 21 days). At the end of the third week, a follow-up survey was conducted to collect information about any adverse events (AE) experience by participants. If no AE were identified or reported, and the participant expressed the willingness to continue using the PR-ENDS device, participants had the option to be enrolled in an extension of the trial. More than 200,000 individual puffs were collected over a two-month period.

Information about the participants' demographics and nicotine history collected from the baseline survey is presented in Table 10, which shows similar puff usage of the PR-ENDS device was observed between female and male users; young adults (e.g., 18-25 years old) and “never smokers” typically recorded low usage of the PR-ENDS, contributing about 4.6% and about 13.8% to the total puffs recorded.

TABLE 10
Summary of participants' demographics and nicotine history (n =
61) and PR-ENDS puff distribution (n = 200,411)

| | Number | Percentage | Number | Percentage |
|---|---|---|---|---|
| | of | of the | of PR-ENDS | of total |
| | participants | population | puffs | puffs |
| Parameters | (n) | (%) | (n) | (%) |
| Sex | | | | |
| Female | 31 | 51% | 98,619 | 49.2% |
| Male | 29 | 48% | 100,899 | 50.3% |
| Prefer not to say | 1 | 2% | 893 | 0.4% |
| Age | | | | |
| 18-25 | 7 | 11% | 9,304 | 4.6% |
| 26-35 | 16 | 26% | 58,099 | 29.0% |
| 36-55 | 33 | 54% | 92,327 | 46.1% |
| ≥56 | 5 | 8% | 40,681 | 20.3% |
| With cigarette | | | | |
| smoking history | | | | |
| Yes | 23 | 38% | 106,647 | 53.2% |
| No | 7 | 11% | 27,607 | 13.8% |
| Did not respond | 31 | 51% | 66,157 | 33.0% |
| Years of | | | | |
| ENDS use | | | | |
| 6 months to 1 year | 4 | 7% | 23,060 | 11.5% |
| 1-5 years | 26 | 43% | 51,306 | 25.6% |
| 6-10 years | 23 | 38% | 94,923 | 47.4% |
| >10 years | 6 | 10% | 30,832 | 15.4% |
| Did not respond | 2 | 3% | 290 | 0.1% |

FIGS. 10A-10B depict a PR-ENDS distribution by device power and by e-liquid nicotine concentration. As shown in FIG. 10A, the high, medium, and low power of the PR-ENDS device contributed 46.4%, 37.4%, and 16.1% respectively, to the total puffs, which indicated that all three power settings were sufficiently utilized (at least 30,000 puffs) by participants, which is further seen in Table 11.

TABLE 11
PR-ENDS puff distribution by power settings (n = 200,411)

| | Number of | Percentage of the |
|---|---|---|
| Device power | puffs (n) | total puffs (%) |
| High | 93,062 | 46.4% |
| Medium | 75,016 | 37.4% |
| Low | 32,333 | 16.1% |

About 60% of the total recorded puffs (n=118,949) contained information on the nicotine concentration of the e-liquids used (shown in Table 12), and the distribution of puffs by nicotine concentration is shown in FIG. 10B. Three (3) mg/ml (27.4%) and 6 mg/ml (27.2%) were recognized as the most prevalent nicotine concentrations for e-liquids used in PR-ENDS devices, followed by 11 mg/ml (12.2%), 18 mg/ml (11.4%), and 14 mg/ml (7.3%). Certain nicotine concentrations were of lower use than that from similar concentrations such as 1 mg/ml (compared to 3 mg/ml) and 10 mg/ml (compared to 11 mg/ml). This may be due to the low availability of such concentrations on the available market or the e-liquid preference of the general population.

TABLE 12
PR-ENDS puff distribution by e-liquid
nicotine concentrations (n = 200,411)

| | Number | Percentage |
|---|---|---|
| e-liquid nicotine | of puffs | of the total |
| concentration | (n) | puffs (%) |
| 0 mg/ml | 5,150 | 2.6% |
| 1 mg/ml | 570 | 0.3% |
| 3 mg/ml | 32,602 | 16.3% |
| 6 mg/ml | 32,333 | 16.1% |
| 10 mg/ml | 3,913 | 2.0% |
| 11 mg/ml | 14,480 | 7.2% |
| 14 mg/ml | 8,695 | 4.3% |
| 16 mg/ml | 4,555 | 2.3% |
| 18 mg/ml | 13,533 | 6.8% |
| 36 mg/ml | 3,118 | 1.6% |
| Blank (no concentration | 81,462 | 40.6% |
| information provided) | | |

As the PR-ENDS device includes three discrete power settings for the purpose of supplying different ranges of wattage to heat e-liquids, the contributed PR-ENDS total puffs were divided by combinations of different power settings and e-liquid nicotine concentrations, as seen in Table 13.

TABLE 13
PR-ENDS puff distribution (%) by the combinations
of device power and nicotine concentration

Device Power

| | Low | Medium | High |
|---|---|---|---|
| Nicotine | power | Power | power |
| concentration | (7-9 watts) | (9-11 watts) | (11-13 watts) |
| 0 mg/ml | 3.9% | 0.4% | 0.0% |
| 1 mg/ml | 0.0% | 0.0% | 0.5% |
| 3 mg/ml | 3.6% | 13.0% | 10.8% |
| 6 mg/ml | 1.9% | 6.2% | 19.1% |
| 10 mg/ml | 0.5% | 1.1% | 1.8% |
| 11 mg/ml | 2.0% | 9.3% | 0.8% |
| 14 mg/ml | 1.1% | 1.7% | 4.6% |
| 16 mg/ml | 0.5% | 1.6% | 1.7% |
| 18 mg/ml | 1.1% | 5.9% | 4.4% |
| 36 mg/ml | 1.4% | 0.1% | 1.2% |

A concise analysis of the information of Table 13 is shown in Table 14, where e-liquid nicotine concentrations are categorized into zero nicotine (0 mg/ml), low nicotine (≤6 mg/ml), medium nicotine (6-14 mg/ml), and high nicotine (≥14 mg/ml).

TABLE 14
PR-ENDS puff distribution (%) by the combinations
of device power and nicotine concentration

Device Power

| | Low | Medium | High |
|---|---|---|---|
| Nicotine | power | Power | power |
| concentration | (7-9 watts) | (9-11 watts) | (11-13 watts) |
| Zero nicotine | 3.9% | 0.4% | 0.0% |
| (0 mg/ml) | | | |
| Low nicotine | 5.4% | 19.3% | 30.4% |
| (≤6 mg/ml) | | | |
| Medium nicotine | 2.5% | 10.4% | 2.6% |
| (6-14 mg/ml) | | | |
| High nicotine | 4.0% | 9.2% | 11.8% |
| (≥14 mg/ml) | | | |

Across all three device powers, low nicotine e-liquid was the most prevalently used nicotine concentration. For zero nicotine e-liquid, the most prevalently used device power is low power. Fewer puffs were generated using medium or high power with the 0 mg/ml e-liquid. For low and high nicotine e-liquids, the most prevalently used device power is high power, followed by medium and low power, while for medium nicotine e-liquids, medium power is the most prevalently used device power. The reason for the observed complex interactions between device power and nicotine concentration may be because that some users may prefer certain power settings for a certain range of nicotine concentrations, such as a relatively high device power with low nicotine e-liquids or vice versa. Other users have opposite preferences such as a high device power with high nicotine e-liquids to reduce craving or a low device power with low/zero nicotine e-liquids to cut back nicotine use. Medium power showed a much higher PR-ENDS usage with medium nicotine e-liquids (compared to low and high power), which may lead to higher consumptions of nicotine in actual use.

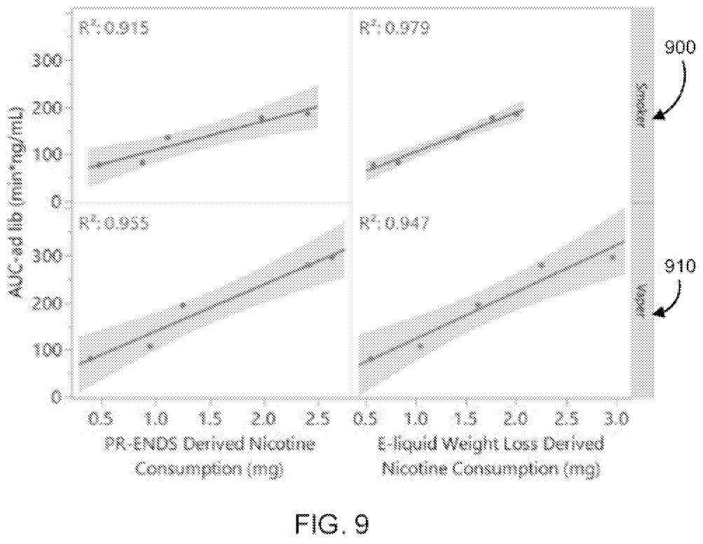

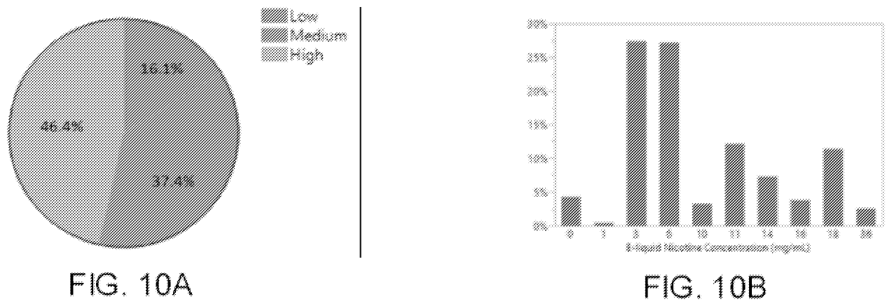

Thus, the PR-ENDS enables naturalistic and noninvasive assessment of puff topography and puffing behaviors, where users can use the device freely with no interference from investigators. The distribution of the 200,411 individual puff data is depicted in FIG. 11A. The puff duration is slightly right-skewed, with mean and median values of 3.44 and 3.10 seconds, respectively. The observed small tail in puff duration distribution (at 10 seconds) can be explained by the automatic power shutdown mechanism built in the PR-ENDS device, which means puffs longer than 10 seconds are not possible.

During actual use, various factors, including device, e-liquid, and user profile, may influence the puff duration. To assess the effect of different factors on PR-ENDS puffs, the measured puff durations were compared by device power, e-liquid nicotine concentration, and nicotine use history (cigarette and ENDS). Given the large puff sample size (>200,000 puffs), the statistical difference in puff duration identified by variable comparisons such as analysis of variance or ANOVA is uninformative (e.g., showing the significant difference with p<0.001 regardless of the selected variables, data not shown). Instead, r² coefficient and Cohen's d (for categorical variable) are better suited for interpreting the magnitude of effect size in statistical analysis.

Based at least in part on the r² coefficients in Table 15, device power, nicotine concentration, and nicotine use history (cigarette and ENDS) all may have small effects (r²<0.10) on PR-ENDS puff duration. As shown in graph 1110 of FIG. 11B, low, medium, and high power yielded puff durations with comparable mean values, although it is recognized in Table 15 that the difference of puff duration between medium and high power is moderately significant (Cohen's d=0.573). Based at least in part on the comparisons shown in graphs 1120, 1130, and 1140 of FIG. 11B, e-liquid nicotine concentration, cigarette smoking history, and years of ENDS use seemed to have no obvious effects on puff duration (r2 coefficient: 0.005-0.060). However, more years of ENDS use is moderately associated with higher puff durations, with the results shown in the differences of puff durations between >10 years and 6 months to 1 year, and between 1-5 years and 6 months to 1 year (graph 1140 of FIG. 11B).

TABLE 15
Summary of effect size (r² coefficient and Cohen's d)
on puff duration by device power, e-liquid nicotine concentration,
and nicotine (cigarette and ENDS) use history

| | r² coefficient | Cohen's d |
|---|---|---|
| Device Power | 0.069 | 0.573 (medium power |
| | | vs. high power) |
| Nicotine | 0.060 | (Not applicable) |
| Concentration | | |
| Cigarette Smoking | 0.005 | (Not applicable) |
| History | | |
| Years of ENDS use | 0.054 | 0.814 (>10 years vs. |
| | | 6 months to 1 year) |
| | | 0.676 (1-5 years vs. |
| | | 6 months to 1 year) |

Based at least in part on Laboratory testing results shown in Table 16, it has been concluded that the nicotine emission (per puff) from PR-ENDS increases with the change of device power from low to medium to high power as well as from low nicotine concentration (3 mg/ml) to high nicotine concentration (12 mg/ml).

TABLE 16
Summary of nicotine emission (average) per puff for PR-ENDS

| | | Nicotine | Average |
|---|---|---|---|
| | | concentration | nicotine |
| | Puff | of e-liquid | emission |
| Device Power | Duration | (mg/ml) | (mg/puff) |
| High | 3 second | 3 | 0.0180 |
| | 3 second | 12 | 0.0952 |
| Medium | 3 second | 3 | 0.0144 |
| | 3 second | 12 | 0.0762 |
| Low | 3 second | 3 | 0.0111 |
| | 3 second | 12 | 0.0406 |

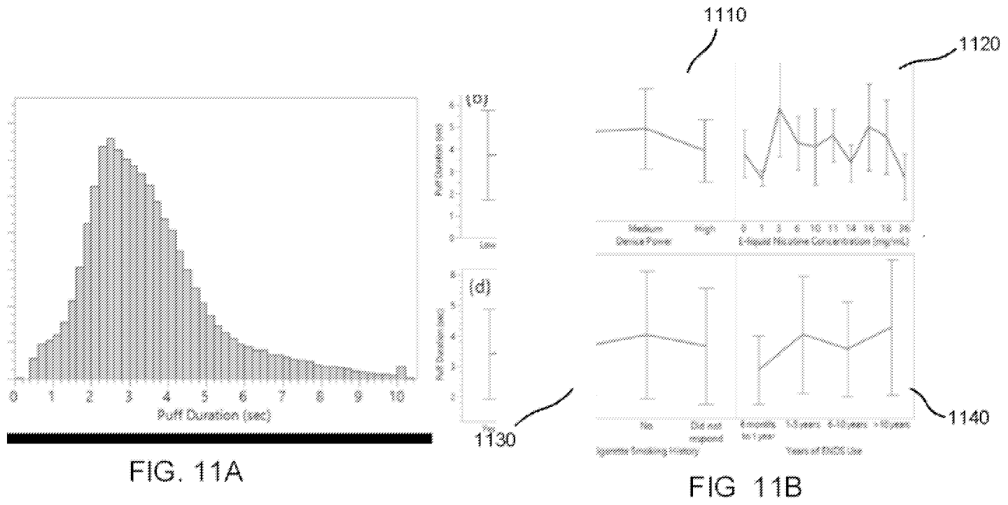

Two assumptions were applied during the estimation of nicotine emissions in PR-ENDS actual use: (1) the nicotine emission is linearly associated with the measured puff duration, and (2) the nicotine emission is linearly associated with the e-liquid nicotine concentration, as shown in FIGS. 12A-12C which depicts a linear regression plotting between nicotine emission per puff and e-liquid nicotine concentrations among high, medium, and low device power. As such, nicotine emission per puff can be estimated using equation (3):

$$( 3 )$$

$$Nicotine emission per puff = \frac{Nicotine {emission}_{Testing}}{\begin{matrix}Puff {Duration}_{Testing} ⨯ E - liquid nicotine {concentration}_{Testing} ⨯ \\ E - liquid nicotine {concentration}_{PR - ENDS} ⨯ Puff {druation}_{PR - ENDS}\end{matrix}}$$

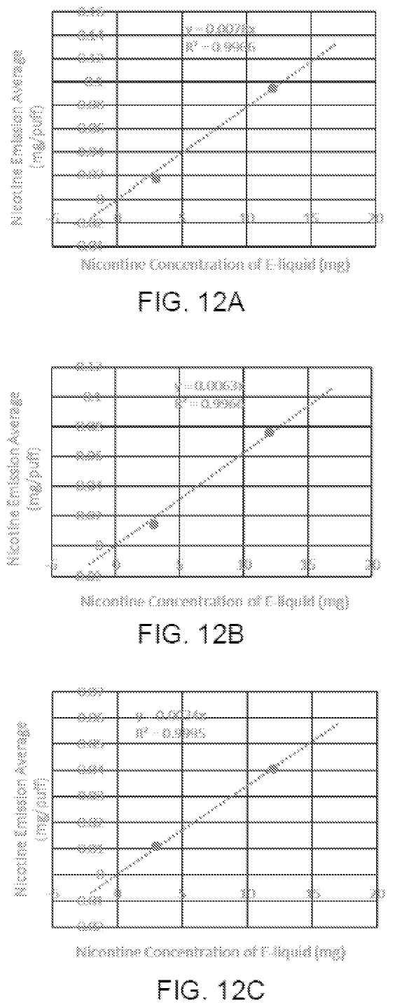

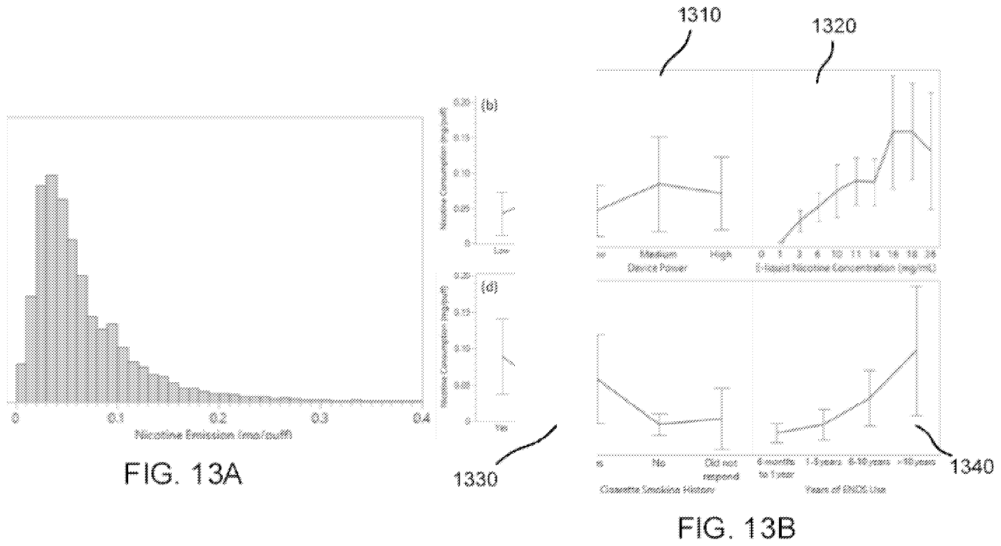

Based at least in part on the 113,797-puff data collected on the PR-ENDS with a record of non-zero nicotine concentrations (as shown in Table 12), the distribution of PR-ENDS derived nicotine emission (per puff) is shown in FIG. 13A. The distribution is right skewed with mean and median values of 0.0648 mg/puff and 0.0508 mg/puff, respectively. Tests on the PR-ENDS have shown that the device yields a nicotine emission of ˜0.0952 mg/puff (as seen in Table 16) when used with 12 mg/ml e-liquid and high power and operated with the Coresta puff regime (55 ml/3 sec/30 ssec). Considering that lower device powers (Low and Medium power) and lower nicotine concentrations of e-liquids (e.g., 3 mg/ml and 6 mg/ml) were prevalently used during actual use, the currently observed PR-ENDS nicotine emissions are deemed reasonable. They are lower than the laboratory testing result and are generally lower than that from commonly used nicotine products such as heated tobacco products and combustible cigarettes.

To assess the effect of different factors on nicotine emissions during PR-ENDS actual use, the calculated values were compared by device power, e-liquid nicotine concentration and nicotine use history (cigarette and ENDS) accordingly. Based at least in part on r² coefficient in Table 17, the effect of device power on nicotine emission is considered small (r2=0.042). As shown in FIG. 13B, medium power yielded the highest average nicotine emission per puff compared to high and low power, and the difference in nicotine emission is moderately significant between medium and low power (Cohen's d>0.5) but not between high and low power (Cohen's d<0.5). Such observations indicated that users preferred applying medium power instead of low or (even) high power for a higher nicotine emission within a single PR-ENDS puff. This can be ascribed to the complex interaction effect of combining different device power and e-liquid nicotine concentrations during the actual use of ENDS, and the previous discussion has suggested that a much higher contribution of using medium nicotine e-liquids was preferably associated with medium device power (Table 14). As a consequence, medium power yielded the highest nicotine emission compared to low and high power.

TABLE 17
Summary of effect size (r² coefficient and Cohen's d) on
nicotine emission per puff by device power, e-liquid concentration,
and nicotine (cigarette and/or ENDS) use history

| | r² coefficient | Cohen's d |
|---|---|---|
| Device Power | 0.042 | 0.666 (medium power |
| | | vs. low power) |
| Nicotine Concentration | 0.422 | Not applicable |
| Cigarette Smoking History | 0.246 | 1.228 (Yes vs. No) |
| | | 1.078 (Yes vs. Did |
| | | not respond) |
| Years of ENDS use | 0.351 | 2.432 (>10 years vs. |
| | | 6 months to 1 year) |
| | | 2.190 (>10 years vs. |
| | | 1-5 years) |
| | | 1.410 (>10 years vs. |
| | | 6-10 years) |
| | | 1.022 (6-10 years vs. |
| | | 6 months to 1 year) |
| | | 0.781 (6-10 years vs. |
| | | 1-5 years) |

In contrast to device power, which may generate a small effect on nicotine emission, e-liquid concentration may render a relatively large effect (r²=0.422), followed by years of ENDS use (r²=0.351) and cigarette smoking history (r²=0.246). Coherent with high r² coefficients, graph 1320 of FIG. 13B showed that a higher nicotine emission per puff is strongly correlated with a higher e-liquid nicotine concentration, especially in the range from 0 mg/ml to 16 mg/ml. For nicotine use history, based at least in part on comparisons shown in graph 1330 and graph 1340 in FIG. 13B, both cigarette smoking and years of ENDS use have moderate correlation effects with nicotine emission per puff. As a result, PR-ENDS users with a cigarette smoking history and more years of ENDS use tended to consume more nicotine per puff with large Cohen's d values presented in Table 17.

The above demonstrates that the PR-ENDS device is capable of measuring actual use puffing behavior as well as their correlations with various use factors in real-world settings. Furthermore, when the device is securely connected to device app with a smartphone or a computer via Bluetooth, the puff data can be uploaded to the cloud for real-time monitoring of product use behavior. Such a feature not only empowers individuals with the awareness to help them quit or cut back their nicotine use, but also provides an effective observation platform for assessing individual and group puffing behaviors and understanding any potential use trends or patterns as proactive post-market surveillance. With the real-time puff data of 58 users collected for 2 months, the daily puff numbers, puff durations, and daily nicotine consumptions for each user can be calculated and the statistics of actual use puffing behavior parameters were summarized in Table 18.

TABLE 18
Summary of actual use puffing behavior characteristics over time by participants (N = 58)

| | Daily puff | | | | | | Daily nicotine | | |
|---|---|---|---|---|---|---|---|---|---|
| | duration (second) | | | Daily puff numbers | | | consumption (mg) | | |
| | | | Coef. of | | | Coef. of | | | Coef. of |
| Participant | Mean (SD) | | Variance | Means (SD) | | Variance | Mean (SD) | | Variance |
| 1 | 1.87 | (0.06) | 3% | 96.67 | (60.80) | 63% | 3.53 | (2.49) | 0.7% |
| 2 | 3.00 | (0.72) | 24% | 184.77 | (102.55) | 56% | 6.68 | (3.76) | 0.56% |
| 3 | 1.73 | (0.43) | 25% | 54.09 | (93.36) | 173% | / | | / |
| 4 | 1.85 | (1.55) | 84% | 67.90 | (87.53) | 129% | 2.33 | (3.00) | 1.29% |
| 5 | 1.52 | (0.00) | 0% | 5.00 | (00.00) | 0% | / | | / |
| 6 | 3.80 | (0.23) | 6% | 189.08 | (31.62) | 17% | 9.60 | (4.38) | 0.46% |
| 7 | 1.84 | (0.26) | 14% | 157.81 | (50.71) | 32% | 16.15 | (7.42) | 0.46% |
| 8 | 2.54 | (0.19) | 7% | 217.46 | (53.61) | 25% | 23.63 | (9.45) | 0.4% |
| 9 | 2.55 | (0.48) | 19% | 364.15 | (106.43) | 29% | / | | / |
| 10 | 2.90 | (1.01) | 35% | 42.75 | (49.48) | 116% | 1.14 | (1.19) | 1.04% |
| 11 | 2.16 | (0.39) | 18% | 124.27 | (79.16) | 64% | 4.27 | (2.65) | 0.62% |
| 12 | 1.96 | (0.14) | 7% | 110.33 | (64.77) | 59% | 2.82 | (1.69) | 0.6% |
| 13 | 4.53 | (0.71) | 16% | 42.67 | (26.56) | 62% | / | | / |
| 14 | 3.87 | (0.16) | 4% | 227.46 | (97.40) | 43% | 12.54 | (5.32) | 0.42% |
| 15 | 2.42 | (0.25) | 1% | 354.33 | (108.50) | 31% | 11.66 | (4.97) | 0.43% |
| 16 | 3.65 | (0.71) | 19% | 43.84 | (55.05) | 126% | 2.75 | (3.60) | 1.31% |
| 17 | 4.68 | (0.40) | 9% | 115.67 | (86.27) | 75% | / | | / |
| 18 | 2.34 | (0.57) | 24% | 14.60 | (14.00) | 96% | 0.50 | (0.49) | 0.99% |
| 19 | 1.83 | (0.00) | 0% | 8.00 | (00.00) | 0% | 0.08 | (0.00) | 0% |
| 20 | 2.45 | (0.19) | 8% | 334.46 | (64.11) | 19% | 24.77 | (6.29) | 0.25% |
| 21 | 3.58 | (0.24) | 7% | 235.94 | (50.76) | 22% | 18.78 | (5.17) | 0.28% |
| 22 | 4.86 | (0.61) | 12% | 412.68 | (132.93) | 32% | 13.54 | (4.60) | 0.34% |
| 23 | 2.36 | (0.15) | 6% | 434.12 | (89.15) | 21% | / | | / |
| 24 | 3.29 | (0.03) | 1% | 27.50 | (22.50) | 82% | 1.71 | (1.41) | 0.82% |
| 25 | 4.40 | (0.31) | 7% | 214.04 | (42.03) | 20% | 36.24 | (6.81) | 0.19% |
| 26 | 5.50 | (1.05) | 19% | 163.00 | (156.93) | 96% | 3.73 | (3.02) | 0.81% |
| 27 | 4.38 | (0.98) | 22% | 22.71 | (26.69) | 118% | 2.13 | (2.55) | 1.2% |
| 28 | 1.51 | (0.12) | 8% | 200.50 | (112.50) | 56% | / | | / |
| 29 | 1.89 | (0.43) | 23% | 185.60 | (133.09) | 72% | 0.67 | (0.29) | 0.44% |
| 30 | 6.19 | (0.45) | 7% | 357.33 | (182.07) | 51% | 11.39 | (8.37) | 0.73% |
| 31 | 3.61 | (0.91) | 25% | 109.52 | (139.03) | 127% | 1.91 | (2.15) | 1.13% |
| 32 | 2.16 | (0.01) | 1% | 290.50 | (03.50) | 1% | 28.29 | (0.87) | 0.03% |
| 33 | 2.64 | (0.28) | 11% | 188.00 | (143.02) | 76% | 13.37 | (10.60) | 0.79% |
| 34 | 1.81 | (0.01) | 1% | 14.50 | (02.50) | 17% | 0.67 | (0.14) | 0.21% |
| 35 | 2.30 | (0.39) | 17% | 285.33 | (142.13) | 50% | / | | / |
| 36 | 2.41 | (0.30) | 13% | 121.43 | (59.04) | 49% | 5.56 | (4.49) | 0.81% |
| 37 | 2.77 | (0.19) | 7% | 372.69 | (196.70) | 53% | / | | / |
| 38 | 3.31 | (0.44) | 13% | 79.67 | (41.63) | 52% | 5.99 | (3.33) | 0.56% |
| 39 | 1.34 | (0.00) | 0% | 217.00 | (00.00) | 0% | 2.17 | (0.00) | 0% |
| 40 | 2.16 | (0.32) | 15% | 374.00 | (267.95) | 72% | 5.22 | (4.08) | 0.78% |
| 41 | 2.35 | (0.32) | 14% | 336.67 | (143.83) | 43% | 21.84 | (10.86) | 0.5% |
| 42 | 2.03 | (0.06) | 3% | 41.00 | (08.00) | 20% | 1.22 | (0.37) | 0.31% |
| 43 | 3.27 | (0.44) | 13% | 455.17 | (255.83) | 56% | 14.47 | (8.90) | 0.62% |
| 44 | 3.93 | (0.35) | 9% | 204.94 | (84.56) | 41% | 29.91 | (13.17) | 0.44% |
| 45 | 4.53 | (0.87) | 19% | 281.52 | (206.77) | 73% | 7.35 | (4.75) | 0.65% |
| 46 | 2.41 | (0.38) | 16% | 23.25 | (14.24) | 61% | 1.08 | (0.89) | 0.82% |
| 47 | 3.80 | (0.47) | 12% | 48.25 | (28.36) | 59% | 1.42 | (0.75) | 0.53% |
| 48 | 2.66 | (0.17) | 6% | 99.22 | (76.53) | 77% | 6.79 | (5.13) | 0.76% |

| | | | | | | | | |
|---|---|---|---|---|---|---|---|---|
| 49 | 0.90 | (0.00) | 0% | 1.00 | (00.00) | 0% | / | / |

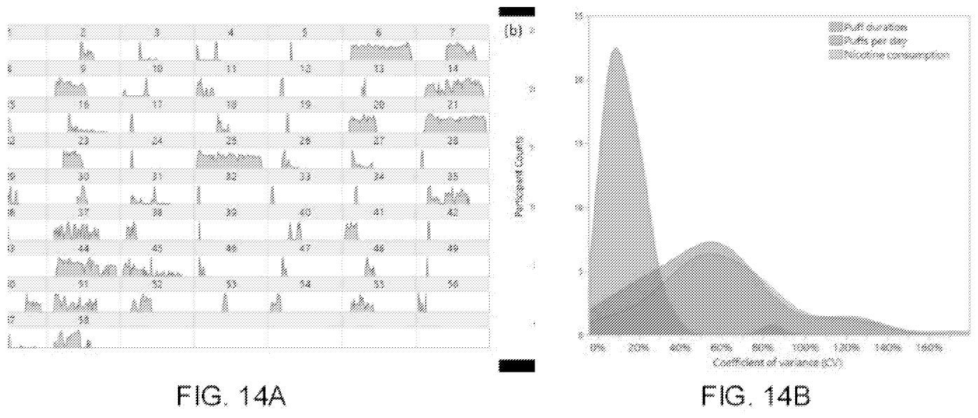

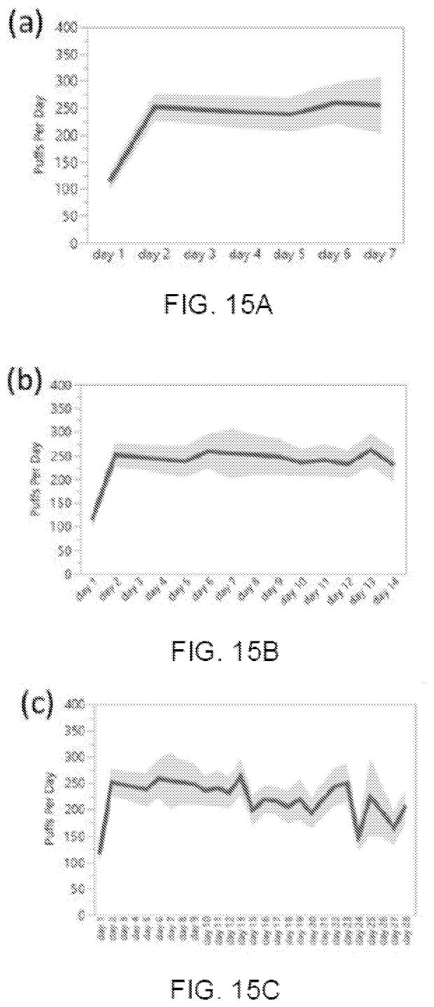

The real-time PR-ENDS use (in daily puff numbers per user) is depicted in FIG. 14A as the normalized plot histogram of puff number vs date. Significant differences in use patterns can be seen between different individuals. For example, certain participants (e.g., participants 6, 21, 25, etc.) continuously used PR-ENDS for more than 50 days with relatively stable use intensity (daily puff numbers), while others tended to use the device somewhat sporadically (3, 4, 16, 46-49, etc.), with following days inactive in product use. Some participants (1-7, 25-28, etc.) used PR-ENDS with less than 250 puffs per day during the actual use, yet others used PR-ENDS more intensively, with more than 400 puffs (22, 23, 43, 54, etc.) recorded per day. Based at least in part on the summarized data shown in Table 18, the puff durations of participants vary from 0.90 seconds to 6.87 seconds; the daily puff numbers vary from 5 puffs to more than 400 puffs; and the daily nicotine consumptions vary from 0.08 mg to 36.24 mg.

Besides the diverse PR-ENDS use patterns in different participants, substantial variabilities of puffing behaviors within the same user profile over time can be found. FIG. 14B depicts the distribution of coefficient of variance (CV) for participants in daily puff numbers, daily average puff duration, and daily nicotine consumptions over their own active period. Most participants had large variances (0-160%) in daily puff numbers and daily nicotine consumptions. However, their daily average puff durations were much less variable, with most participants' CVs located in the range of 0-40%. The significant variability of puff number and nicotine consumption within each individual participant represented the actual use situation in real-world settings. It is significant that users' puffing behavior, aside from puff duration, did not present a consistent use format, but rather evolved dynamically over time. For example, users had higher puff numbers and nicotine consumptions on certain days yet had lower puff numbers and nicotine consumptions on other days. However, most participants' daily puff durations during actual use were relatively stable, and no significant variance was identified over the observation period.

From the perspective of group behavior assessment, longitudinal observations of PR-ENDS puffing behaviors over time (e.g., three weeks or longer) should be treated as strong indicators to interpret the product specific nicotine addiction potential and abuse liability. Specifically, when the observed participant group is being treated as a cohort, their first recorded day of using PR-ENDS can be considered as day 1 in the longitudinal observation. Puffs per day thus can be calculated by taking puff numbers from active users in each day into account. Puff duration per day can be calculated by averaging the puff duration of the active users in each day. Nicotine consumption per day can be obtained by calculating accumulated puffs with nicotine consumption in each puff of active users in each day.

Puffs per day at the population level can be calculated by averaging the number of puffs form the active users in each day. Specifically, the number of puffs for user j at day k was recorded by PR-ENDS and uploaded to the cloud. Due to the fact that different participant was enrolled into the observation session at different date, day 1 for user j was recognized as the first date in which user j's puff data was observed. The puffs per day at the population level (for the active users) at day k was then calculated based at least in part on equation (4). The number of active users at day k refers to the number of users whose puff data was observed at day k.

$$Puffs Per {day}_{@ Day K} = \frac{{Σ}_{i} Number of {Puffs}_{User j @ Day K}}{Number of Active {Users}_{@ Day K}} \qquad ( 4 )$$

With the puffs per day at day k calculated, the puffs per day over time was plotted in FIG. 14A. The standard error of puffs per day was calculated for the active users at day k.

Puff duration per day at the population level can be calculated by averaging the puff duration from the active users in each day. Specifically, the puff duration for user j at day k was calculated based at least in part on equation (5) with PR-ENDS recorded data. Due to the fact that different participants were enrolled into the observation session at different dates, day 1 for j was recognized as the first date in which user j's puff data was observed.

$$Puff {Duration}_{User j @ Day K} = \frac{{Σ}_{i} Puff {Duration}_{Puff i , User j @ Day K}}{Number of {Puffs}_{User j @ Day K}} \qquad ( 5 )$$

Puff duration per day at the population level (for all active users) at day k was then calculated on equation (6). The number of active users at day k refers to the number of users whose puff data was observed at day k.

$$Puff {Duration}_{@ Day K} = \frac{{Σ}_{i} Puff {Duration}_{User j @ Day K}}{Number of Active {Users}_{@ Day K}} \qquad ( 6 )$$

With the puff duration per day at day k calculated, the puff duration per day over time was plotted in FIG. 14B. The standard error of puff duration per day was calculated for the active users at day k.

Nicotine consumption per day at the population level can be calculated by taking the nicotine emission per puff and the associated number of puffs from the active users in each day. The calculation of nicotine emission per puff is described in equation (3) and the number of puffs from active users in each day is described in reference to equation (4).

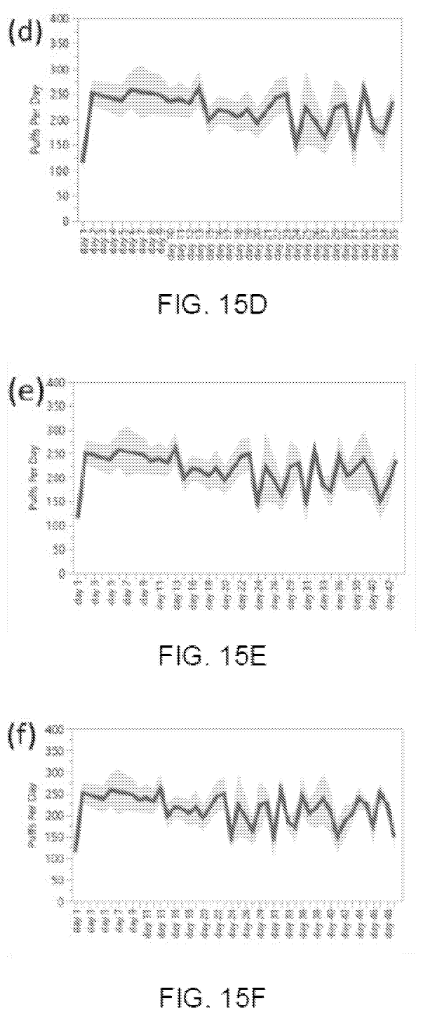

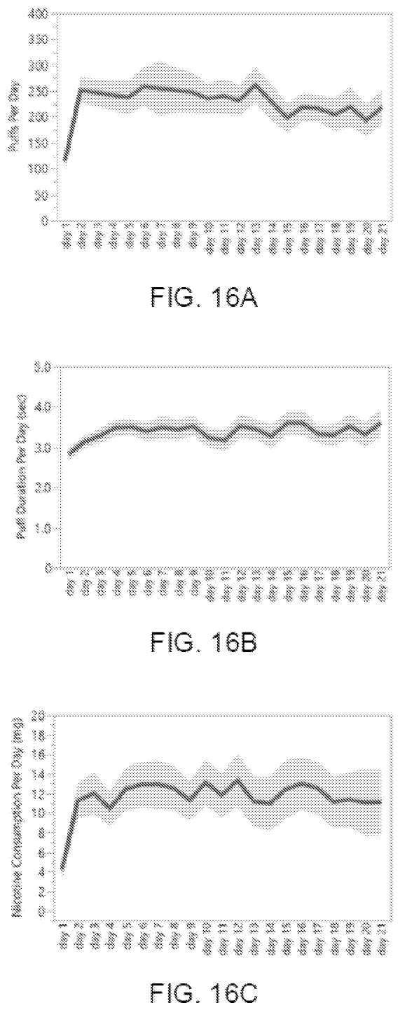

For puffs per day, as shown in FIG. 16A, the participant group initiated the actual use of PR-ENDS devices with about 120 puffs on the first day (day 1), and the group quickly adapted to “normal operation” of ˜250 puffs per day after one to two days (day 2-3). The puffs per day value then stabilized over time until the end of the third week. Over the timespan of three weeks, the participant cohort consistently used the PR-ENDS device with no observable increase in puffs per day over time. This finding is consistent with the examination on daily puff numbers for each individual that no obvious ramp-up trends were identified in FIG. 14A. FIGS. 15A-15F further assessed the robustness of this observation, with a similar trend identified for users who used PR-ENDS for one, two, four, five, six, and seven weeks. The PR-ENDS use pattern showed an initial low puff number on the first day, followed by a quick increase and plateauing of puffs per day due to adaptation to habitual use.

For puff duration per day, as shown in FIG. 16B, the observed group initiated the use with an average puff duration of ˜2.8 seconds at day 1 and the value gradually increased and plateaued to ˜3.5 seconds after about 5-7 days. This puff duration trend was consistently maintained until the end of the third week (21 days). The stabilized puff duration was found to be consistent with the puff duration ((3.44±1.65 sec) shown in FIG. 11A. The low intra-individual variability in puff durations in FIG. 14B also validated the consistent puff durations. This further indicates that after acclimatization to the use of the PR-ENDS, participants consistently used the device with limited abuse tendency and with no significant increase in puff durations after 1 week and beyond.

For nicotine consumption per day of the cohort group, as shown in FIG. 16C, it is observed that participants started using PR-ENDS with an average daily nicotine consumption of ˜4.2 mg on day 1. User group then quickly adapted to “normal operation”, with nicotine consumption per day increasing and plateauing at ˜12 mg/day after 1-2 days. Nicotine consumption per day then stabilized until the end of the third week. The trend of nicotine consumption over time observed was almost the same as puffs per day, which indicates either that user did not change e-liquid nicotine concentration over the observation period or that the user did not update their app record after such a change.

It was found that PR-ENDS device was primarily consumed by existing nicotine product users who are well past young adulthood (age range), which is likely due to the design feature of PR-ENDS as a complicated open e-cigarette system that entails smoking or vaping experience. It is expected that nicotine naïve users are not primarily interested in using this device. As an ENDS product that requires e-liquid refill and wattage adjustment (three discrete power settings: low, medium, and high), a diverse range of e-liquid nicotine concentrations as well as a complex interactive effect between e-liquid nicotine concentrations and device powers were recognized based at least in part on information collected from PR-ENDS. For example, high power was recognized as the most prevalently used power setting when the device was combined with low (≤6 mg/ml) and high (≥14 mg/ml) nicotine e-liquids; medium power of the device contributed much more puffs with medium (6-14 mg/ml) nicotine e-liquids; while low power is predominantly used for zero nicotine e-liquid (0 mg/ml). Such observation highlighted the importance of being able to provide a wide range of device powers and e-liquid nicotine concentrations during e-cigarette actual use for reducing nicotine craving and smoking transition.

The PR-ENDS collected information showed a reasonable distribution of puff duration (3.44±1.65 seconds) based at least in part on >200,000 individual puff data. The observed puff duration during actual use correspond to the value proposed in aerosol testing protocols. The value aligned well with the puff duration data published from other e-cigarette use behavior studies, where puffs were found to last from 2 to 4 seconds during the actual. The statistical significance can consistently be seen when PR-ENDS puff durations were compared under different device power, nicotine concentration, or nicotine history (data not shown). However, calculations on the effect size (r² coefficient and Cohen's d) unveiled that none of the factors above yielded a significant change in puff duration during actual use.

Considering that the puff topography and PR-ENDS specific information in estimating nicotine consumption (e.g., device power, e-liquid, etc.) was collected in situ, it is viable to evaluate the PR-ENDS based nicotine emission estimates, as well as to assess the implications on nicotine use by comparing nicotine emissions under different factors. For example, it was observed that medium power was associated with the highest average nicotine emission per puff compared to low or high power. This is probably due to the fact that medium power was much more frequently used with medium (6-14 mg/ml) and high (≥14 mg/ml) nicotine e-liquids and that higher nicotine concentrations are prone to yield higher nicotine emissions in product use. The current finding is that the change in nicotine emission per puff is not directly proportional to the increase of PR-ENDS device power from low to medium to high. Instead, the free selection of e-liquid nicotine concentrations rendered the actual use inevitably more complex (e.g., medium power associated with the highest nicotine emissions instead of high power).

In addition, higher nicotine concentrations, cigarette smoking history, and more years of ENDS use all led to higher nicotine emission per puff with relatively significant effect size, even though the associated puff durations are relatively comparable and with a small effect size. As identified from previous research, e-cigarette users may attempt compensatory puffing patterns and nicotine self-titrations, with puff number and puff durations being lower while liquid and nicotine consumption being higher when they used e-cigarettes with a higher power setting. However, in the current actual use observation, we found that the compensatory puff pattern is not significant (small effect on puff duration) while nicotine emission was strongly correlated with various factors (large effect on nicotine emission). Such contrasting result brought further contextualization to the identified confounding effects here, as the selection of device power, e-liquid nicotine concentration, puff topography, and nicotine consumptions are all interrelated to each other and are affected by the puffing behavior and nicotine history during the actual use. The findings may demonstrate that choices in nicotine concentration and device power settings are important influences on the behavior of e-cigarette users. The more “subconscious” influence of puff duration shows much lower variability, meaning that users' self-titration of nicotine consumption is likely to be a conscious choice.

With puff data recorded in real-time, time course of PR-ENDS puffing behavior at both individual and population levels may be considered. When the puff data is viewed by each user profile over the period of actual use, different product use patterns in daily puff numbers can be recognized such that some participants had a consistent trend in puffs per day with PR-ENDS, while others chose to use the device sporadically without continuity of use over time. Further, the calculation of the coefficient of variance from puffs per day and nicotine consumption for the same PR-ENDS user showed a large variability over their own active period. These observations, taken together, highlighted the considerable unpredictability in both inter- and intra-individual actual use of puffing behaviors and emphasized the importance of discovering puffing behavior patterns at the individual level with real-time feedback.

The time course of the entire PR-ENDS cohort consistently showed a quick adaptation to device use followed by a consistent use pattern. As presented in FIGS. 16A-16C, the participant group initiated the use of PR-ENDS with about 120 puffs and 2.8-second puff duration on day 1, and then quickly adapted to normal operation of 250 puffs and 3.5-second puff duration in 1-2 days and 5-7 days, respectively. After 1 week, the daily puff number and puff duration of the user group stabilized and plateaued until the end of day 21. The initial increase in daily puff numbers and puff durations observed for PR-ENDS is consistent with findings where users tended to prolong their puffs using a 10 W PR-ENDS device filled with 6 mg/ml e-liquid over 5 consecutive days (the study period). Considering the similarity of the device power and e-liquid nicotine concentration between the two investigated PR-ENDS devices, the identification of the same trend in increasing the use of PR-ENDS is not surprising. However, the observation duration in the current study was at least 21 days, which is much longer than 5 days in the previous study, allowing us to identify trends over a relatively long term. This included the consistent use pattern of puff number and puff duration after acclimatization to the use of PR-ENDS post 1 week of use.

The observed trend of the PR-ENDS estimated daily nicotine consumption rendered a similar use pattern over time as the daily puff number in the user group. The users' daily nicotine consumption was observed with an initial increase step in 1-2 days followed by consistent use for at least three weeks or 21 days. The eventual stabilized daily nicotine consumption was found to be ˜12 mg/day. A direct comparison of daily nicotine consumption between PR-ENDS and other nicotine products is challenging. However, based at least in part on a previous data on the daily intake of nicotine from cigarette smoking with an average nicotine consumption of 37.6 mg, the nicotine consumption calculated from PR-ENDS is ˜30% of the nicotine intake from smoking per day. It should be noted that the nicotine consumption in the current observation is rather theoretical and based at least in part on laboratory testing results, while the previous study of nicotine intake was conducted with blood specimen analysis. Further studies are warranted to investigate the overall nicotine consumption from PR-ENDS users (including smoking and other nicotine products) during actual use.

Some key strengths of the actual use behavior assessment include: (1) serving as an observational behavior assessment conducted in real-world conditions that systematically examined the puff topography and puffing behavior at both individual and population levels. (2) identifying the complex interactions between device power and e-liquid nicotine concentrations in e-cigarette actual use and their effects on puff topography and use behaviors. (3) revealing that a significant variability of puffing behaviors exists between different users and within the same individual user over time. A quick adaptation pattern (an increase of puff number and puff duration followed by stabilized product use for at least 3 weeks) may be observed when the cohort was assessed as a whole.

### Additional Considerations

It should be noted that the term “example”, “exemplary”, or the like, as used herein to describe various embodiments or arrangements is intended to indicate that such embodiments or arrangements are possible examples, representations, and/or illustrations of possible embodiments or arrangements (and such term is not intended to connote that such embodiments or arrangements are necessarily crucial, extraordinary, or superlative examples).

The arrangements disclosed herein have been described with reference to drawings. The drawings illustrate certain details of specific arrangements that implement the systems, methods and programs disclosed herein. However, describing the arrangements with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” and/or “module” may include hardware structured to execute the functions disclosed herein. In some arrangements, each respective “circuit” may include machine-readable media for configuring the hardware to execute the functions disclosed herein. The circuit may be embodied as one or more circuitry components including, but not limited to, processing circuitry, network interfaces, peripheral devices, input devices, output devices, sensors, etc. In some arrangements, a circuit may take the form of one or more analog circuits, electronic circuits (e.g., integrated circuits (IC), discrete circuits, system on a chip (SOCs) circuits, etc.), telecommunication circuits, hybrid circuits, and any other type of “circuit.” In this regard, the “circuit” may include any type of component for accomplishing or facilitating achievement of the operations disclosed herein. For example, a circuit as disclosed herein may include one or more transistors, logic gates (e.g., NAND, AND, NOR, OR, XOR, NOT, XNOR, etc.), resistors, multiplexers, registers, capacitors, inductors, diodes, wiring, and so on).

The “circuit” and/or “module” may also include one or more processors communicatively coupled to one or more memory or memory devices. In this regard, the one or more processors may execute instructions stored in the memory or may execute instructions otherwise accessible to the one or more processors. In some arrangements, the one or more processors may be embodied in various ways. The one or more processors may be constructed in a manner sufficient to perform at least the operations disclosed herein. In some arrangements, the one or more processors may be shared by multiple circuits (e.g., circuit A and circuit B may comprise or otherwise share the same processor which, in some example arrangements, may execute instructions stored, or otherwise accessed, via different areas of memory). Alternatively or additionally, the one or more processors may be structured to perform or otherwise execute certain operations independent of one or more co-processors. In other example arrangements, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some arrangements, the one or more processors may be external to the apparatus, for example the one or more processors may be a remote processor (e.g., a cloud-based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud-based server). To that end, a “circuit” as disclosed herein may include components that are distributed across one or more locations.

An exemplary system for implementing the overall system or portions of the arrangements might include a general-purpose computing computer in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some arrangements, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other arrangements, the volatile storage media may take the form of RAM, TRAM, ZRAM, etc. Combinations of the above are also included within the scope of machine-readable media. In this regard, machine-executable instructions comprise, for example, instructions and data which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example arrangements disclosed herein.

It should also be noted that the term “input devices,” as disclosed herein, may include any type of input device including, but not limited to, a keyboard, a keypad, a mouse, joystick, touch sensitive screen or other input devices performing a similar function. Comparatively, the term “output device,” as disclosed herein, may include any type of output device including, but not limited to, a computer monitor, printer, facsimile machine, or other output devices performing a similar function.

It should be noted that although the diagrams herein may show a specific order and composition of method operations, it is understood that the order of these operations may differ from what is depicted. For example, two or more operations may be performed concurrently or with partial concurrence. Also, some method operations that are performed as discrete operations may be combined, operations being performed as a combined operation may be separated into discrete operations, the sequence of certain processes may be reversed or otherwise varied, and the nature or number of discrete processes may be altered or varied. The order or sequence of any element or apparatus may be varied or substituted according to alternative arrangements. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure may be accomplished with standard programming techniques with rule-based logic and other logic to accomplish the various database searching operations, correlation operations, comparison operations and

While this specification contains many specific implementation details, these should not be construed as limitations on the scope of any arrangement or of what may be claimed, but rather as descriptions of features specific to particular implementations of particular arrangements. Certain features described in this specification in the context of separate implementations can also be implemented in combination in a single implementation. Conversely, various features described in the context of a single implementation can also be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a subcombination or variation of a subcombination.

What is claimed is:

1. A controller, comprising:
a processor; and
a memory storing instructions configured to cause said processor to:
receive emission test data of a test aerosolization system (TAS) corresponding to at least a test aerosolizer power or a range of test aerosolizer powers of a test aerosolizer of said test aerosolization system and a test concentration or a range of test concentrations;
receive operation data of a user aerosolization system (UAS) corresponding to an operation duration based at least in part on an input signal from a user, said operation data comprising at least a concentration of a consumable included in said user aerosolization system and an aerosolization power level of an aerosolizer of said user aerosolization system during said operation duration;
estimate an emission per operation duration based at least in part on said operation data and said emission test data; and
generate a signal indicative of said estimated emission per operation duration.

2. The controller of claim 1, wherein said processor is configured to estimate said emission per operation duration based at least in part on a test emission of said emission test data, a test concentration, a test operation duration of said emission test data, and one or more aerosolization generation conditions.

3. The controller of claim 2, wherein said processor is configured to estimate said emission per operation duration based at least in part on said concentration and said operation duration.

4. The controller of claim 3, wherein said processor is configured to estimate said emission per operation duration using said following equation:
$${emission}_{operation duration UAS , i} = \frac{{emission}_{TAS}}{{concentration}_{TAS} ⨯ operation {duration}_{TAS}} ⨯ {concentration}_{UAS , i} ⨯ operation {duration}_{UAS , i}$$
wherein said emission_{operation duration UAS, i} is said emission per operation duration for an operation duration i, emission_{TAS} is said test emission, concentration_{TAS} is said test concentration, operation duration_{TAS} is said test operation duration, concentration_{UAS, i} is said concentration for said operation duration i, and operation duration_{UAS} is said operation duration i.

5. The controller of claim 1, wherein said processor is further configured to:
estimate a total emission of said aerosolization system based at least in part on a emission per operation duration and a total number of operation durations since an initial operation of said user aerosolization system by said user.

6. The controller of claim 1, wherein said processor is further configured to:
estimate an accumulated consumption over a period of time using said following equation:
$$accumulated {consumption}_{t 0 \to t 1} = \begin{matrix}\sum \\ {operation}_{UAS , i} , t 0 \to t 1\end{matrix} consumable {emission}_{{operation}_{UAS , i}}$$

7. The controller of claim 6, wherein said processor is further configured to:
estimate a consumable absorption in human based using said following equation:
$$consumable absorption in human {body}_{t 0 \to t 1} = Coefficient C ⨯ accumulated {consumption}_{t 0 \to t 1}$$
wherein Coefficient C is a value in a range of approximately 0.001 to 0.80, inclusive.

8. The controller of claim 1, wherein said instructions further cause said processor to:
display operation information on a user device, wherein said operation information comprises at least said emission per operation duration.

9. The controller of claim 1, further comprising:
a network interface communicably coupled to a communication network, wherein said communication network is communicably coupled to said user aerosolization system.

10. The controller of claim 9, wherein communication network is communicably coupled to at least one database, wherein said at least one database stores said emission test data.

11. The controller of claim 1, wherein said operation duration corresponds to a duration of said input signal.

12. The controller of claim 11, wherein:
said input signal corresponds to activation of a switch included in said user aerosolization system, and
said operation duration corresponds to an amount of time said switch is activated by said user.

13. The controller of claim 11, wherein:
said input signal corresponds to draw pressure from a pressure sensor, and
the operation duration corresponds to an amount of time said draw pressure remains above a predetermined threshold.

14. The controller of claim 1, wherein said user aerosolization system is a nicotine aerosolization system.

15. A method for determining an aerosolization emission, comprising:
receiving, by a controller, emission test data of a test aerosolization system (TAS) based at least in part on a test aerosolizer power or a range of a test aerosolizer powers of a test aerosolizer of said test aerosolization system and a test concentration or a range of test concentrations;
receiving, by said controller, operation data of a user aerosolization system (UAS) corresponding to an operation duration based at least in part on an input signal from a user, said operation data comprising at least a concentration of a consumable included in said user aerosolization system and an aerosolizer power level of an aerosolizer of said user aerosolization system during said operation duration;
estimating, by said controller, a emission per operation duration based at least in part on said operation data and emission test data; and
generating, by said controller, a signal indicative of said estimated emission per operation duration.

16. The method of claim 15, wherein estimating said emission per operation duration is based at least in part on a test emission of said emission test data, a test concentration, a test operation duration of said emission test data, and one or more aerosolization general conditions.

17. The method of claim 16, wherein estimating said emission per operation duration is based at least in part on said concentration and said operation duration.

18. The method of claim 17, wherein said controller is configured to estimate said emission per operation duration using said following equation:
$${emission}_{operation duration UAS , i} = \frac{{emission}_{TAS}}{{concentration}_{TAS} ⨯ operation {duration}_{TAS}} ⨯ {concentration}_{UAS , i} ⨯ usage {duration}_{UAS , i}$$
wherein said emission_{operation duration UAS, i} is said emission per operation duration for an operation duration i, emission_{TAS} is said test emission, concentration_{TAS} is said test concentration, operation duration_{TAS} is said test operation duration for said operation duration i, concentration_{UAS} is said concentration, and operation duration_{UAS} is said operation duration i.

19. The method of claim 15, further comprising:
estimating a total emission of said aerosolization system based at least in part on a number of emission per operation duration and a total number of operation durations since an initial operation of said user aerosolization system by said user.

20. The method of claim 15, further comprising:
estimate an accumulated consumption over a period of time using said following equation:
$$accumulated {consumption}_{t 0 \to t 1} = \begin{matrix}\sum \\ {operation}_{UAS , i} , t 0 \to t 1\end{matrix} consumable {emission}_{{operation}_{UAS , i}}$$

21. The method of claim 20, further comprising:
estimate a consumable absorption in human based using said following equation:
$$consumable absorption in human {body}_{t 0 \to t 1} = Coefficient C ⨯ accumulated {consumption}_{t 0 \to t 1}$$
wherein Coefficient C is a value in a range of approximately 0.001 to 0.80, inclusive.

22. The method of claim 19, wherein said signal further indicates said total emission of said aerosolization system.

23. The method of claim 15, wherein said operation duration corresponds to a duration of said input signal.

24. An aerosolization device, comprising:
an aerosolizer configured to aerosolize a consumable; and
a controller comprising (i) one or more processors and (ii) one or more memories storing computer-executable instructions that, when executed, cause said one or more processors to:
(a) receive operation data of said aerosolizer correspond to an operation duration based at least in part on an input signal from a user, said operation data comprising at least a concentration of said consumable included in said aerosolizer and an aerosolization power level of said aerosolizer during said operation duration,
(b) estimate an emission per operation duration for said aerosolizer based at least in part on said operation data, and
(c) cause said aerosolizer to suspend aerosolization of said consumable based at least in part on said emission per operation duration.

25. The aerosolization device of claim 24, wherein said emission per operation duration is estimated based at least in part on said concentration and said operation duration.

26. The aerosolization device of claim 24, wherein said computer-executable instructions, when executed, further cause said one or more processors to:
estimate a total emission of said aerosolizer based at least in part on an emission per operation duration and a total number of operation durations since an initial operation of said aerosolizer by said user.

27. The aerosolization device of claim 24, wherein said computer-executable instructions, when executed, further cause said one or more processors to:
estimate an accumulated consumption over a period of time using said following equation:
$$accumulated {consumption}_{t 0 \to t 1} = \begin{matrix}\sum \\ {operation}_{UAS , i} , t 0 \to t 1\end{matrix} consumable {emission}_{{operation}_{UAS , i}}$$

28. The aerosolization device of claim 27, wherein said computer-executable instructions, when executed, further cause said one or more processors to:
estimate a consumable absorption in human based using said following equation:
$$consumable absorption in human {body}_{t 0 \to t 1} = Coefficient C ⨯ accumulated {consumption}_{t 0 \to t 1}$$
wherein Coefficient C is a value in a range of approximately 0.001 to 0.80, inclusive.

29. The aerosolization device of claim 24, wherein said computer-executable instructions, when executed, further cause said one or more processors to:
display operation information on a user device, wherein said operation information comprises at least said emission per operation duration.

30. The aerosolization device of claim 24, further comprising:
a network interface communicably coupled to a communication network, wherein said communication network is communicably coupled to said aerosolizer.

31. The aerosolization device of claim 30, wherein communication network is communicably coupled to at least one database, wherein said at least one database stores emission test data.

32. The aerosolization device of claim 24, wherein said operation duration corresponds to a duration of said input signal.

33. The aerosolization device of claim 32, wherein:
said input signal corresponds to activation of a switch included in said aerosolizer, and
said operation duration corresponds to an amount of time said switch is activated by said user.

34. The aerosolization device of claim 32, wherein:
said input signal corresponds to draw pressure from a pressure sensor, and
said operation duration corresponds to an amount of time said draw pressure remains above a predetermined threshold.

35. The aerosolization device of claim 24, wherein said aerosolizer is a nicotine aerosolization system.

36. The aerosolization device of claim 24, wherein said consumable comprises one or more of: 5-MeO-DMT (5-methoxy-N,N-dimethyltryptamine), Psilocybin, MDMA (3,4-Methylenedioxymethamphetamine), DMT (N,N-Dimethyltryptamine), LSD (Lysergic acid diethylamide), Ketamine, or esketamine.

37. The aerosolization device of claim 24, wherein said consumable comprises one or more of: 4-isobutyl-alpha-methylphenylacetic acid (IBUPROFEN®), acetylsalicylic acid (ASPIRIN®), (S)-(+)-2-(6-methoxy-2-naphthyl)propionic acid (NAPROXEN®), hydrocortisone, diphenhydramine (BENADRYL®), chlorpheniramine maleate (CLARITIN®), doxylamine succinate (UNISOM®), cetirizine dihydrochloride, melatonin, 1-tryptophan, 5-hydroxy-1-tryptophan, 4-acetamidophenol (TYLENOL®), 1-phenylephrine, guaiacol glycerol ether (MUCINEX®), salbutamol hemisulfate, humic acid, or other medicaments.

38. The aerosolization device of claim 24, wherein said consumable comprises one or more of phosphatidylcholine, cranberry powder, echinacea extract, feverfew extract, flaxseed extract, flaxseed extract, honeysuckle extract, white willow bark extract, lotus leaf extract, organic ginger extract, ashwagandha, bilberry, hops, horse chestnut, green coffee bean, luteolin, milk thistle, olive leaf, Radix isatidis, Rhodiola rosea, rose hip, tongkat ali, quercetin, American ginseng, Andrographis, astragalus, black pepper extract (95% piperine), boswelia serrata, cassia seed, chamomile, cinnamon bark, dandelion, dong quai root, elderberry, fennel seed, fenugreek, Ginkgo biloba, ginseng root, gotu kola, green tea extract (50% EGCG), green tea extract (50% polyphenols), hawthorn berry, lemon balm, lemon powder, licorice root, luo han guo, magnolia, marshmallow root, papaya fruit, passion flower, peppermint, pine bark, pomegranate, red clover, resveratrol, rhodiola extract (3% salidroside), schisandra, slippery elm bark, spirulina, tart cherry, theobromine, tribulus terrestris, turmeric extract (95% curcuminoids), valerian root, white mulberry, wild cherry, or wild jujube.

39. The aerosolization device of claim 24, wherein said consumable comprises one or more of Agomelatine, Duloxetine, Imipramine, or other depression medications.

40. The aerosolization device of claim 24, wherein said consumable comprises one or more of Apomorphine, Levodopa, or other Parkinson's medication.

41. The aerosolization device of claim 24, wherein said consumable comprises one or more of Loxapine, Chlorpromazine, or other schizophrenic or bipolar disorder medications.

42. The aerosolization device of claim 24, wherein said consumable comprises one or more of Dihydroergotamine, Sumatriptan, Prochlorperazine, Metoclopramide, Lidocaine, Duloxetine, or other migraine medications.

43. The aerosolization device of claim 24, wherein said consumable comprises one or more of caffeine, tea, or other stimulants.

44. The aerosolization device of claim 24, wherein said consumable comprises one or more of Ciprofloxacin, Norfloxacin, Balofloxacin, Rufloxacin, Fleroxacin, Gatifloxacin, Levofloxacin, Moxifloxacin, Ofloxacin, Sparfloxacin, Pefloxacin, Nadifloxacin, Clofazimine, Homosulfamine, Metronidazole, Ampicillin, Azithromycin, Tetracycline, Vancomycin, Amikacin, Cefadroxil, Aztreonam, Tobramycin, or other antibiotics
